(19) United States
(12) Patent Application Publication
(10) Pub. No.: US 2014/0302567 A1
(43) Pub. Date: Oct. 9, 2014

(54) METHODS AND COMPOSITIONS USING LIGNOLYTIC ENZYMES AND MEDIATORS TO REDUCE AND REFORM LIGNIN CONTENTS IN LIGNOCELLULOSIC BIOMASS

(71) Applicant: THE UNIVERSITY OF GEORGIA RESEARCH FOUNDATION, INC, Athens, GA (US)

(72) Inventors: Qingguo Huang, Fayetteville, GA (US); Sudeep S. Sidhu, Griffin, GA (US)

(73) Assignee: THE UNIVERSITY OF GEORGIA RESEARCH FOUNDATION, INC, Athens, GA (US)

(21) Appl. No.: 14/351,296

(22) PCT Filed: Oct. 11, 2012
(86) PCT No.: PCT/US12/59710

§ 371 Date: Apr. 11, 2014

Related U.S. Application Data
(60) Provisional application No. 61/547,314, filed on Oct. 14, 2011.

(51) Int. Cl. C12P 19/04; C12N 9/02

(52) U.S. Cl. CPC C12P 19/04; C12N 9/0061
USPC 435/101; 435/192; 435/189

(57) ABSTRACT

Embodiments of the present disclosure include methods of treating a biomass, in particular lignocellulosic biomass (e.g., switch grass, sweet sorghum, miscanthus, pine wood, corn stover, and the like), under appropriate conditions to effectively reduce and/or reform the lignin contents in lignocellulosic biomass without significantly reducing its sugar contents.

CLAIM OF PRIORITY TO RELATED APPLICATION

This application claims priority to co-pending U.S. provisional application entitled “METHODS AND COMPOSITIONS USING LIGNOLYTIC ENZYMES TO REDUCE AND REFORM LIGNIN CONTENTS IN LIGNOCELLULOSIC BIOMASS” having Ser. No.: 61/547,314, filed on Oct. 14, 2011, which is entirely incorporated herein by reference.

### BACKGROUND

Lignocellulosic biomass is the most abundant renewable resource on the earth, consisting mostly of agricultural wastes, forestry residues and energy crops. It is mostly composed of cellulose, hemicellulose, and lignin. Cellulose is a polysaccharide linked by beta-1,4-glycosidic bonds which can be digested by cellulase and beta-glucosidase to glucose, a fermentable sugar for bioethanol production. Hemicellulose, however, is a highly branched short polymer composed of xylose, arabinose, glucose, galactose, and mannose. Unlike cellulose, hemicellulose is more easily hydrolyzed into monomeric sugars, of which xylose can also be utilized for bioethanol production. Lignin is a complex polyphenolic polymer in lignocellulose which can greatly impede enzymatic hydrolysis for fermentable sugars. The main procedure for bioethanol production from biomass can be divided into the following three steps: 1) pretreatment of biomass; 2) enzymatic hydrolysis to produce fermentable sugars; 3) anaerobic fermentation of sugars to bioethanol. Bioethanol yields are directly dependent on the yield of fermentable sugars available from hydrolysis of pretreated biomass. Thus, a pretreatment method capable of disrupting recalcitrant lignocellulosic structures would be helpful for bioethanol production.

### SUMMARY

Embodiments of the present disclosure include methods of treating a biomass, in particular lignocellulosic biomass (e.g., switch grass, sweet sorghum, miscanthus, pine wood, corn stover, and the like).

In an embodiment, a method of treating a biomass, among others, includes: contacting a lignocellulosic biomass with an enzyme and optionally a mediator; mixing the lignocellulosic biomass with the enzyme and optionally the mediator at about room temperature for a time period; and modifying the content of lignin in a lignocellulosic biomass.

In an embodiment, a composition, among others, includes: an enzyme and a mediator. In an embodiment, the enzyme is selected from the group consisting of: laccase, lignin peroxidase, horseradish peroxidase, manganese peroxidase, tyrosinase, and a combination thereof. In an embodiment, the mediator is selected from the group consisting of: catechol, guaiacol, ABTS, violuric acid, 1-hydroxy-benzotriazole (HBT), veratryl alcohol and a combination thereof.

Other systems, methods, features, and advantages will be, or become, apparent to one with skill in the art upon examination of the following drawings and detailed description. It is intended that all such additional structures, systems, methods, features, and advantages be included within this description, be within the scope of the present disclosure, and be protected by the accompanying claims.

### BRIEF DESCRIPTION OF THE DRAWINGS

Many aspects of this disclosure can be better understood with reference to the following drawings. The components in the drawings are not necessarily to scale, emphasis instead being placed upon clearly illustrating the principles of this disclosure.

FIG. 1.1 illustrates an outline of the experimental procedures. (A): premature switchgrass plant in the field; (B): short pieces (1 cm-1.5 cm) of switchgrass; (C): solid state fermentation in flasks; (D): enzyme extraction using citrate-phosphate buffer; (E): pretreated switchgrass; (F): switchgrass powder for enzymatic hydrolysis (the left plate contained pretreated switchgrass and the right with untreated one); (G): enzymatic hydrolysis in vials (the left three vials contained pretreated switchgrass and the right with untreated ones); (H): The crude extract containing co-product enzymes.

FIG. 1.2 illustrates the oven dry weight (ODW) of untreated and fungal pretreated switchgrass after various periods of cultivation time.

FIG. 1.3 illustrates the chemical composition of untreated or fungal treated switchgrass for various cultivation days. (A): Structural sugars (Gray Bar: glucan, and green Bar: xylan); (B): Acid soluble lignin; (C): Acid insoluble lignin; and (D): Ash.

FIG. 1.4 illustrates the profiles of ligninolytic (A) and hydrolytic enzymes (B) activity in the crude extracts after various periods of cultivation time. In FIG. 1.4A, gray bar: laccase, green bar: lignin peroxidase, blue bar: manganese peroxidase. In FIG. 1.4B, green bar: β-glucosidase. Cellulase and xylanase were not detected in any extract.

FIG. 1.5 illustrates pictures of the outer surface of untreated (A) and fungal pretreated switchgrass stems (B: 18-d; C: 36-d).

FIG. 1.6 illustrates: (A): Enzymatic hydrolysis of untreated and fungal pretreated switchgrass for various periods of cultivation time. Cellulase (Lot#110M1456v) was used at the dosage of 8 U/g solid biomass. (B): Effect of enzyme dosage on enzymatic hydrolysis of untreated and fungal pretreated switchgrass for 36-d. Cellulase (Lot#110M1456v) was used at the dosage of 9, 12.6, 22.5, 30.3, 38, 46, 60, or 70 U/g solid biomass. (C): Enzymatic hydrolysis curve of untreated and fungal pretreated switchgrass for 36-d. Cellulysin cellulase (Cat#219466) was used at the dosage of 40 FPU/g solid biomass.

FIG. 2.1 illustrates the extractive-free acid-soluble lignin content (L_{S}, 8.1A) and acid-insoluble lignin content (L_{I}, 8.1B) of sweet sorghum after 24 h of enzymatic treatment in a 20 mL reaction mixture with seven different levels of mediator ABTS 0, 0.13, 0.25, 0.31, 0.63, 1.25, and 1.88 mM with and without laccase at activity 10 units mL⁻¹. Values are means of three replicates and error bars represent standard deviation. Bars with the same letter are not considered to be statistically different according to Fisher's protected LSD at α=0.05.

FIG. 2.2 illustrates the extractive-free acid-soluble lignin content (L_{S}, 8.2A) and acid-insoluble lignin content (L_{I}, 8.2B) of switchgrass after 24 h of enzymatic treatment in a 20 mL reaction mixture with seven different levels of mediator ABTS 0, 0.13, 0.25, 0.31, 0.63, 1.25, and 1.88 mM with and without laccase at activity 10 units mL⁻¹. Values are means of three replicates and error bars represent standard deviation. Bars with the same letter are not considered to be statistically different according to Fisher's protected LSD at α=0.05.

FIG. 2.3 illustrates the extractive-free acid-soluble lignin content (L_{S}, 8.3A) and acid-insoluble lignin content (L_{I}, 8.3B) of sweet sorghum after 24 h of enzymatic treatment in a 20 mL reaction mixture with seven different levels of mediator HBT 0, 0.13, 0.25, 0.31, 0.63, 1.25, and 1.88 mM with and without laccase at activity 10 units mL⁻¹. Values are means of three replicates and error bars represent standard deviation. Bars with the same letter are not considered to be statistically different according to Fisher's protected LSD at α=0.05.

FIG. 2.4 illustrates the extractive-free acid-soluble lignin content (L_{S}, 8.4A) and acid-insoluble lignin content (L_{I}, 8.4B) of switchgrass after 24 h of enzymatic treatment in a 20 mL reaction mixture with seven different levels of mediator HBT 0, 0.13, 0.25, 0.31, 0.63, 1.25, and 1.88 mM with and without laccase at activity 10 units mL⁻¹. Values are means of three replicates and error bars represent standard deviation. Bars with the same letter are not considered to be statistically different according to Fisher's protected LSD at α=0.05.

FIG. 2.5 illustrates the extractive-free acid-soluble lignin content (L_{S}, 8.5A) and acid-insoluble lignin content (L_{I}, 8.5B) of sweet sorghum after 24 h of enzymatic treatment in a 20 mL reaction mixture with five different levels of mediator VA 0, 0.31, 0.63, 1.25, and 1.88 mM with and without laccase at activity 10 units mL⁻¹. Values are means of three replicates and error bars represent standard deviation. Bars with the same letter are not considered to be statistically different according to Fisher's protected LSD at α=0.05.

FIG. 2.6 illustrates the extractive-free acid-soluble lignin content (L_{S}, 8.6A) and acid-insoluble lignin content (L_{I}, 8.6B) of switchgrass after 24 h of enzymatic treatment in a 20 mL reaction mixture with five different levels of mediator VA 0, 0.31, 0.63, 1.25, and 1.88 mM with and without laccase at activity 10 units mL⁻¹. Values are means of three replicates and error bars represent standard deviation. Bars with the same letter are not considered to be statistically different according to Fisher's protected LSD at α=0.05.

FIG. 2.7 illustrates the measured and calculated dry mass loss from sweet sorghum (8.7A) and switchgrass (8.7B) biomass after 24 h of laccase treatment (10 units mL⁻¹) in a 20 mL reaction mixture with two different levels of mediators ABTS, HBT, and VA at 1.25, and 1.88 mM concentration.

FIG. 2.8A illustrates Table 1, which describes the extractive-free acid-soluble lignin (L_{S}), acid-insoluble lignin (L_{I}), and total lignin (L_{T}) in sweet sorghum and switchgrass after 24 h of treatment with laccase enzyme at 0, 2, 5, 10, and 20 units mL⁻¹ activity in a 20 mL reaction mixture.

FIG. 2.8B illustrates Table 2, which describes the total lignin content (extractive-free) in sweet sorghum and switchgrass biomass after 24 h treatment with laccase-mediator system. The 20 mL reaction mixture consisted of laccase at 10 units mL⁻¹ activity with one of the three mediators; ABTS, HBT, and VA at different concentrations.

FIG. 2.8C illustrates Table 3, which describes extractive-free acid-soluble lignin (L_{S}), acid-insoluble lignin (L_{I}), and total lignin (L_{T}) in sweet sorghum after 24, 48, and 72 h of enzymatic treatment. The 20 mL reaction mixture consisted of laccase at 10 units mL⁻¹ activity with one of the three mediators; ABTS, HBT, and VA at different concentrations.

FIG. 2.8D illustrates Table 4, which describes the extractive-free acid-soluble lignin (L_{S}), acid-insoluble lignin (L_{I}), and total lignin (L_{T}) in switchgrass after 24, 48, and 72 h of enzymatic treatment. The 20 mL reaction mixture consisted of laccase at 10 units mL⁻¹ activity with one of the three mediators; ABTS, HBT, and VA at different concentrations.

FIG. 2.8E illustrates Table 5, which describes the extractive-free total structural sugar content (S_{T}) of sweet sorghum and switchgrass biomass after 24 h of enzymatic treatment. The 20 mL enzymatic treatment mixture consisted of the three mediators; ABTS, HBT, and VA at concentration of 0.63, 1.25, and 1.88 mM along with laccase enzyme at 10 units mL⁻¹.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of biology, chemistry, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by volume, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequences where this is logically possible.

It must be noted that, as used in the specification and the appended claims, the singular forms “a,” “an,” and “the” include plural referents unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

### Definitions

“Lignocellulosic biomass” can include as-products, by-products, and/or residues of the forestry and agriculture industries that include lignocellulose. Biomass includes, but is not limited to, algae, plants, trees, crops, crop residues, grasses, forest and mill residues, wood and wood wastes, fast-growing trees, and combinations thereof, that include lignocellulose. In particular, biomass can include sweet sorghum, switchgrass, miscanthus, pine wood, and corn stover.

As used herein, “degrade” or “degrading” with respect to biomass indicates that the enzyme and mediator are able to break-down portions of the chemical structure of the biomass including lignin-containing components or otherwise act to reduce the amount (measured by weight, thickness, or other measureable variable) of biomass and/or lignin content of the biomass as compared to a sample not treated with the enzyme and mediator.

### Discussion

Lignocellulose, as one of the most abundant organic sources, has been considered as a potential raw material for biofuel and other chemical products. Lignocellulosic materials contain cellulose and hemicellulose, bound together by lignin. Both cellulose and hemicellulose are built up from long chain sugar monomers, which, after pretreatment and hydrolysis, can be converted into bioethanol or other value-added products.

Embodiments of the present disclosure describe the use of different lignolytic enzymes (e.g., laccase, lignin peroxidase, horseradish peroxidase, and the like), and optionally in combination with a mediator (e.g., ABTS, HBT, violuric acid, and the like), under appropriate conditions effectively reduces and reforms lignin contents in lignocellulosic biomass without significantly reducing its sugar contents. In addition, embodiments of the present disclosure include combinations of enzymes and mediators that result in effective lignin reduction for different biomasses such as switch grass and sweet sorghum. In an embodiment, the methods and compositions of the present disclosure can be used as an alternative process to pretreat lignocellulose for biofuel and chemical product production. In an embodiment, the method has little or does not cause sugar loss, produces less fermentation inhibiting products, and/or is environmentally friendly.

Embodiments of the present disclosure include methods of treating a biomass, in particular lignocellulosic biomass (e.g., switch grass, sweet sorghum, miscanthus, and pine wood, and the like). In an embodiment, the method can be conducted using a solid state fermentation process. In general, the method of treating a biomass is a pretreatment of the biomass to modify the content of the lignin. One or more enzymes, and optionally one or more mediators, are contacted with the lignocellulosic biomass and mixed. In an embodiment, one or more enzymes can be added to the biomass sequentially or simultaneously. In an embodiment, the enzyme and the mediator can be combined in a single composition or system and mixed with the biomass. In an embodiment, the enzyme and the mediator can be added to the biomass simultaneously or sequentially.

After a time period, the content of the lignin is modified (e.g., reduced). In an embodiment, the time period can be about 6 hours or more, about 12 hours or more, about 18 hours or more, about 24 hours or more, about 30 hours or more, or about 36 hours or more. In general, the method is conducted under atmospheric temperature and pressure. In an embodiment, the temperature can be about 0° C. to 100° C. or about 25° C. to 45° C. In an embodiment, the pressure is about 0.1 to 10 atm or about 0.8 to 1.2 atm. In an embodiment, the ratio of the enzyme, to the mediator, to the lignocellulosic biomass is about 1:1:100 to 1:100,000:100,000,000 or about 1:10:1,000 to 1:1,000:1,000,000.

In an embodiment, modifying can include reducing the content of lignin in the lignocellulosic biomass relative the amount originally present in the lignocellulosic biomass. In embodiment, the content of the lignin can be reduced by about 15% or more, about 20% or more, about 25% or more, about 30% or more, or about 35% or more, relative to the original lignin content in the biomass. In an embodiment, while modifying the content of the lignin, the methods substantially (e.g., about 90% or more, about 95% or more, about 98% or more, or about 99% or more) maintain the cellulose and hemicellulose content relative to the cellulose and hemicellulose content prior to the exposure to the enzyme and mediator. In an embodiment, the modifying can include an enzymatic hydrolysis of the lignocellulosic biomass that is about 35% or more after about a 24 hour or more time period, relative to no treatment with the enzyme and the mediator

In an embodiment, the enzyme functions to solubilize and/or mineralize lignin. In an embodiment, the enzyme preferably attacks lignin over cellulose or hemicellulose. In an embodiment, the enzyme can include laccase, lignin peroxidase, horseradish peroxidase, manganese peroxidase, tyrosinase, or a combination thereof. In an embodiment, the enzyme can be about 1/100 to 1/100,000,000 or about 1/10,000 to 1/10,000,000, of the mass of the lignocellulosic biomass.

As used herein, reference to “laccase” (EC 1.10.3.2) can refer to “isolated laccase” and/or” laccase” that is not separated from the source organism. In particular, the enzyme is laccase. In an embodiment, the laccase enzyme can be from white rot fungi (e.g., the white rot species Trametes versicolor and available from Sigma-Aldrich) or from Wuxi AccoBio Biotech, Inc. (Wuxi, China). Other possible sources of the laccase enzyme include, but are not limited to, other natural sources of laccase enzyme as well as another cell or organism, such as, for example, e. coli, that is adapted to produce laccase (e.g., genetically engineered by transformation with a construct containing a gene for laccase). As used herein the term “isolated laccase” or “isolated laccase enzyme” refers to a laccase enzyme that has been separated from its biological source (e.g., white rot fungi). An isolated laccase may or may not be purified (e.g., free from other environmental contaminants, microbial secretes, or deactivated organisms), but it is separated from the source organisms or the source organisms have been deactivated.

Lignin peroxidase (EC 1.11.1.14) or manganese peroxidase (EC 1.11.1.13) can be purchased from Sigma Aldrich or produced by fermentation with white rot fungi, such as Phanerochaete chrysosporium. Horseradish peroxidase (EC 1.11.1.7) can be purchased from Sigma Aldrich, for example as the lyophilized Type I powder, or it can be obtained in crude form by extracting horseradish, or minced/crushed horseradish can be used directly to offer peroxidase activity. Tyrosinase (1.14.18.1) is a copper-containing enzyme present in plant and animal tissues that catalyzes the production of melanin and other pigments from tyrosine by oxidation. It is also available from commercial source, e.g., Sigma Aldrich, or can be obtained through tissue extraction in crude forms.

In an embodiment that includes a mediator, the mediator functions to improve the efficiency of the enzyme. In general, a mediator is a compound used by the enzyme in reactions to break down lignin in the biomass. In particular, the mediator can help electron transfer during enzyme catalysis, and/or alleviate enzyme inactivation, and thus enhance the efficiency of the enzyme. A mediator of the selected enzyme (e.g., laccase) may be included to aid reactions catalyzed by the enzyme leading to degradation of lignin in the biomass. In an embodiment, the mediator may include catechol, guaiacol, ABTS, violuric acid, 1-hydroxy-benzotriazole (HBT), veratryl alcohol, or a combination thereof. In an embodiment, the mediator is about 1/1 to 1/100,000 or about 1/10 to 1/10,000 of the mass of the lignocellulosic biomass.

Enzymatic pretreatment has the capability to selectively decompose lignin over cellulose and hemicellulose in lignocellulosic biomass. A fraction of lignin should degrade into CO₂, while the rest undergoes significant structural changes. The disrupted lignocellulosic structures and the added porosity on pretreated biomass improves the efficiency in the hydrolysis step to yield more fermentable sugars and reduces the formation of side products that negatively impact the fermentation step to produce bioethanol.

Enzymatic pretreatment has some advantages in terms of sustainability and recyclability. The temperature required for the process is much lower than most physicochemical approaches, thus considerably reducing energy input. Another advantage is that the spent substrate can be potentially recycled for other uses, e.g., animal feed, fertilizer, etc., because the entire process is environmentally friendly. When the pretreatment was done using solid phase fermentation with enzyme-producing fungi, the enzymes can be recovered as value-adding co-products.

### EXAMPLES

Now having described the embodiments of the present disclosure, in general, the examples describe some additional embodiments of the present disclosure. While embodiments of the present disclosure are described in connection with the examples and the corresponding text and figures, there is no intent to limit embodiments of the present disclosure to these descriptions. On the contrary, the intent is to cover all alternatives, modifications, and equivalents included within the spirit and scope of embodiments of the present disclosure.

### Example 1

### Brief Introduction

We in this study investigated fungal pretreatment of switchgrass involving solid state fermentation (SSF) to improve saccharification and simultaneously produce enzymes as co-products. The results revealed that the fungus Pycnoporus sp. SYBC-L3 can significantly degrade lignin and enhance enzymatic hydrolysis efficiency. After a 36-d cultivation period, a nearly 30% reduction in lignin content was obtained without significant loss of cellulose and hemicellulose, while a considerable amount of laccase, as high as 6.3 U/g, was produced. After pretreatment, pores on switchgrass surface were observed using scanning electron microscopy (SEM). The enzymatic hydrolysis efficiency for the switchgrass with 36-d pretreatment was about 50% greater than the untreated one. Our results suggest that solid-state fungal cultivation may be a good method for switchgrass pretreatment, which can simultaneously achieve high efficiency of enzymatic hydrolysis and production of some useful enzymes for other industrial utilization.

### Introduction

As known petroleum reserves are gradually depleted, the need of new alternatives to petroleum-based fuels becomes increasingly urgent. Renewable bioenergy sources have drawn the attention of researchers due to the abundance of available raw materials and their reduced environmental impact. Biofuels, such as bioethanol and biodiesel, have become a research hot spot among researchers worldwide. Biofuels can play an important role in helping lessen the impacts of climate change, improving national security, and protecting the environment, and thus have become increasingly important to the global energy supply. One of the most promising bioenergy strategies is to produce bioethanol and biodiesel using grain crops, such as corn and canola. However, this approach will compete with the food supply, potentially leading to a global food crisis (Li and Khraisheh, 2010). To avoid a conflict over farmland, food, edible oil and sugars, some alternatives such as lignocellulosic biomass or agro-wastes can be used as good sources for renewable energy production (Balan et al., 2008).

Large quantities of biomass produced every year can greatly contribute to bioenergy production. At present, some lignocellulosic biomasses have been employed for bioenergy production, e.g., pinewood (Wang et al., 2011), canola residue (George et al., 2010), and switchgrass (Yang et al., 2009). Among lignocellulosic biomasses used for biofuel, switchgrass has become the material of choice due to its high yield, high nutrient-use efficiency and wide geographic distribution (McLaughlin and Walsh, 1998). In addition, switchgrass stands out for its great advantages in conservation of water and soil and grassland improvement, and thus can be viewed as one of the bioenergy feedstocks with the highest potential.

Lignocellulosic biomass is the most abundant renewable resource on the earth, consisting mostly of agricultural wastes, forestry residues and energy crops. It is mostly composed of cellulose, hemicellulose, and lignin. Cellulose is a polysaccharide linked by beta-1,4-glycosidic bonds which can be digested by cellulase and beta-glucosidase to glucose, a fermentable sugar for bioethanol production. Hemicellulose, however, is a highly branched short polymer composed of xylose, arabinose, glucose, galactose, and mannose. Unlike cellulose, hemicellulose is more easily hydrolyzed into monomeric sugars, of which xylose can also be utilized for bioethanol production (Matsushika et al., 2009). Lignin is a complex polyphenolic polymer in lignocellulose which can greatly impede enzymatic hydrolysis for fermentable sugars. The main procedure for bioethanol production from biomass can be divided into the following three steps: 1) pretreatment of biomass; 2) enzymatic hydrolysis to produce fermentable sugars; 3) anaerobic fermentation of sugars to bioethanol (Faga et al., 2010). Bioethanol yields are directly dependent on the yield of fermentable sugars available from hydrolysis of pretreated biomass. Thus, a pretreatment method capable of disrupting recalcitrant lignocellulosic structure is critical for bioethanol production (Pallapolu et al., 2011).

Various physical, chemical, or combined methods have been used to date for the pretreatment of lignocellulosic biomass to disrupt recalcitrant structures. The commonly used methods include dilute acid pretreatment (Esteghlalian et al., 1997), microwave-assisted alkali pretreatment (Hu and Wen, 2008), steam pretreatment (Ewanick and Bura, 2011), hydrothermolysis (Faga et al., 2010), and ionic liquid (Li et al., 2010). These pretreatment methods, while disrupt recalcitrant structures, often alter biomass chemical composition, and further affect subsequent hydrolysis. Most of these methods are performed under severe reaction conditions or using acid and alkali, which are often energy consuming and/or generating new environmental pollutants. Therefore, a mild, low-cost, effective and environmentally friendly biotreatment is advantageous over physical or chemical means (Zeng et al., 2011). Utilizing white-rot fungi with solid state fermentation (SSF) is a promising new pretreatment method (Taniguchi et al., 2005; Taniguchi et al., 2010). SSF is an effective method to degrade lignin and improve biomass saccharification. Currently, white-rot fungi are the only known microorganisms with the capability of effectively degrading lignin due to their powerful extracellular lignin-degrading enzymes (Eggert et al., 1997).

Some typical white-rot fungi, such as Pleurotus ostreatus (Taniguchi et al., 2005) and Ceriporiopsis subvermispora (Wan and Li, 2010) have been shown to degrade lignin. It is also known that lignin peroxidase (LiP), manganese peroxidase (MnP) and laccase are the three main ligninolytic enzymes involved in delignification. Genus Pycnoporus has been considered as one of the most efficient laccase-producing model organisms (Eggert et al., 1997). However, studies evaluating the efficiency of fungal pretreatment by genus Pycnoporus on switchgrass remain scarce. Furthermore, we are unaware of any reports on the evaluation of white-rot fungi for simultaneously reducing lignin content and generating useful enzymes as co-products during biomass pretreatment. Considering that some biomass loss always occur during the fungal pretreatment, the co-products generated during the pretreatment process maybe a compensating or value-adding mechanism to make it practical by reducing the overall cost of bioethanol production.

In this study, we employed a previously isolated white-rot fungus, Pycnoporus sp. SYBC-L3, to pretreat switchgrass with solid state fermentation. The factors that we have examined included disruption of recalcitrant structures, changes in chemical composition, amounts of various enzymes obtained in the crude extracts as co-products, and the efficiency of enzymatic hydrolysis after pretreatment. The main objectives of this study were: (1) to evaluate the efficiency of enzymatic hydrolysis of swtichgrass biomass after fungal pretreatment and (2) to evaluate the potential to produce useful enzymes as co-products during fungal treatment of switchgrass biomass.

### Materials and Methods

### Chemicals, Biomass, Enzymes, and Operating Process

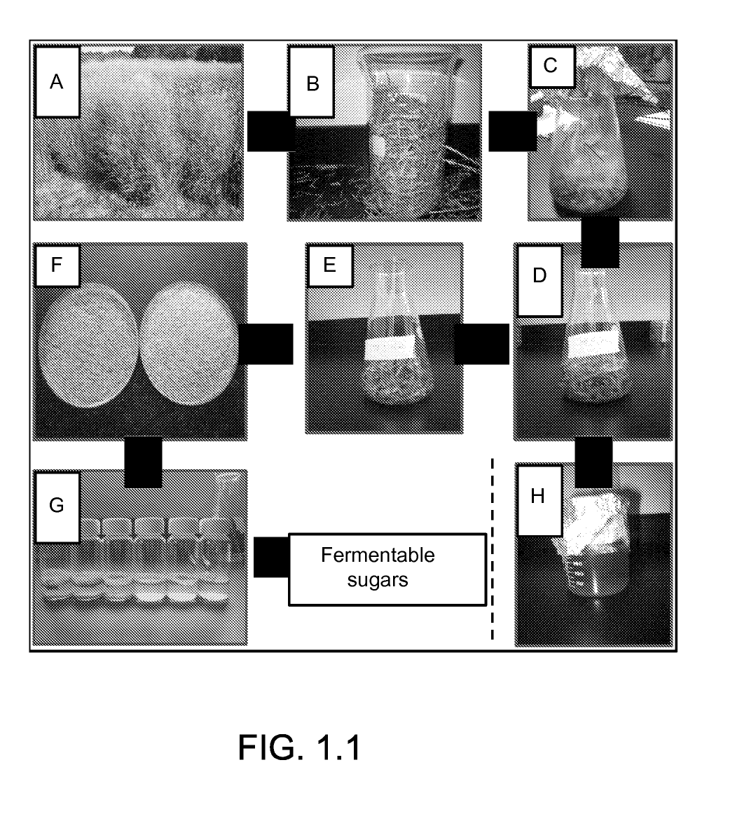

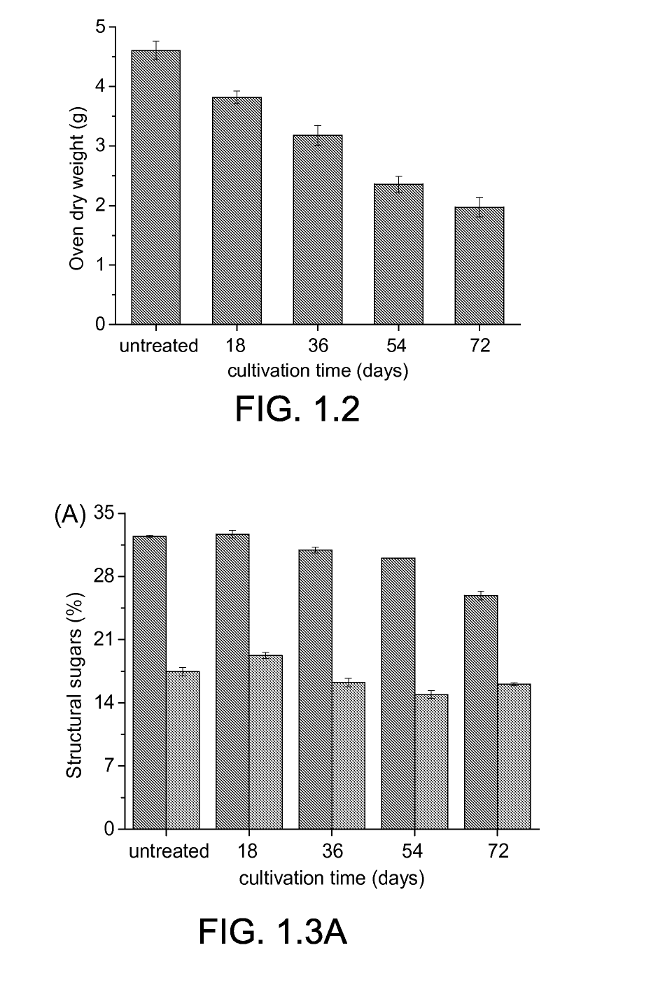

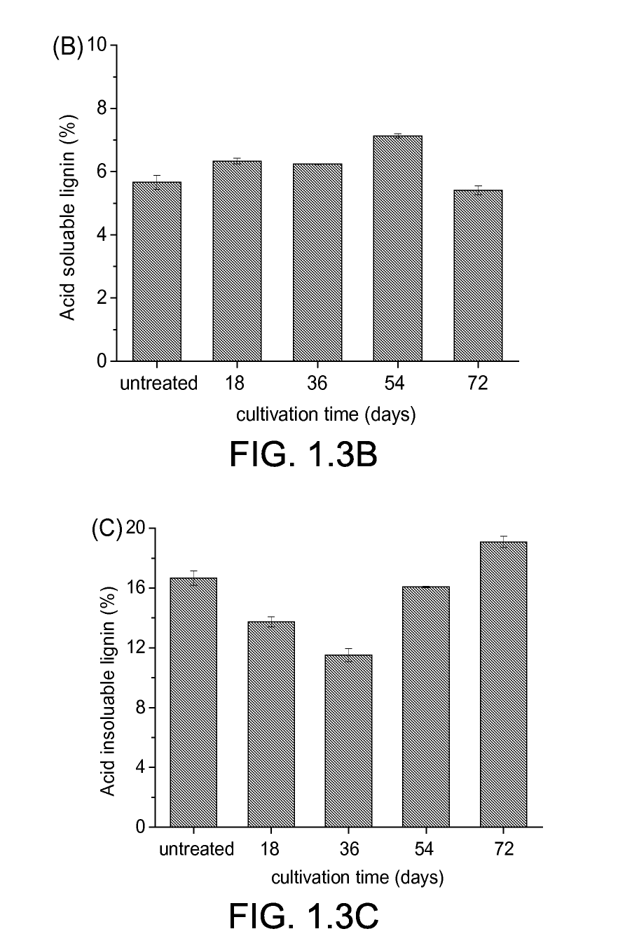

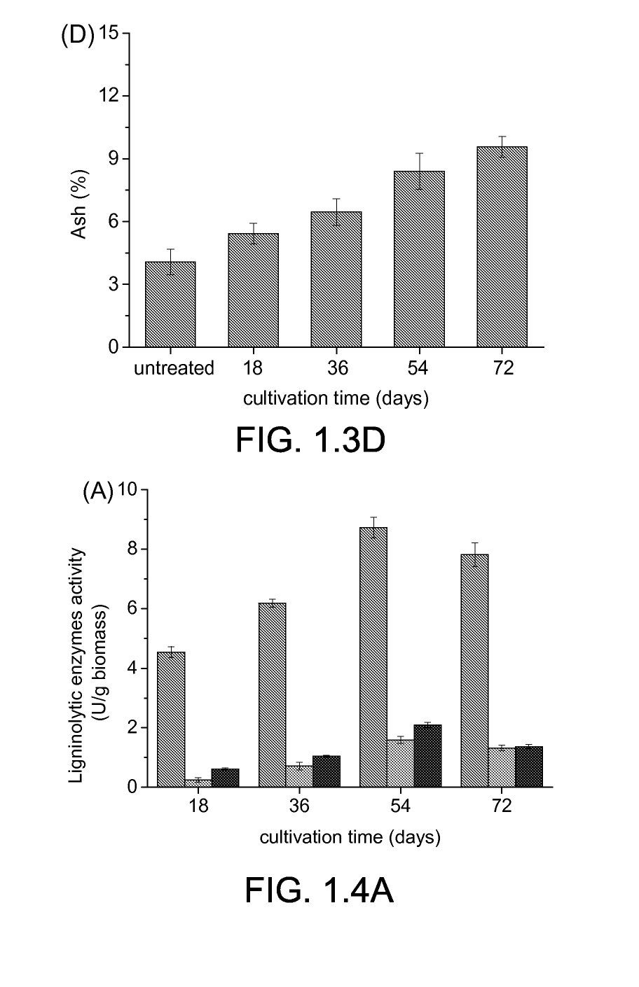

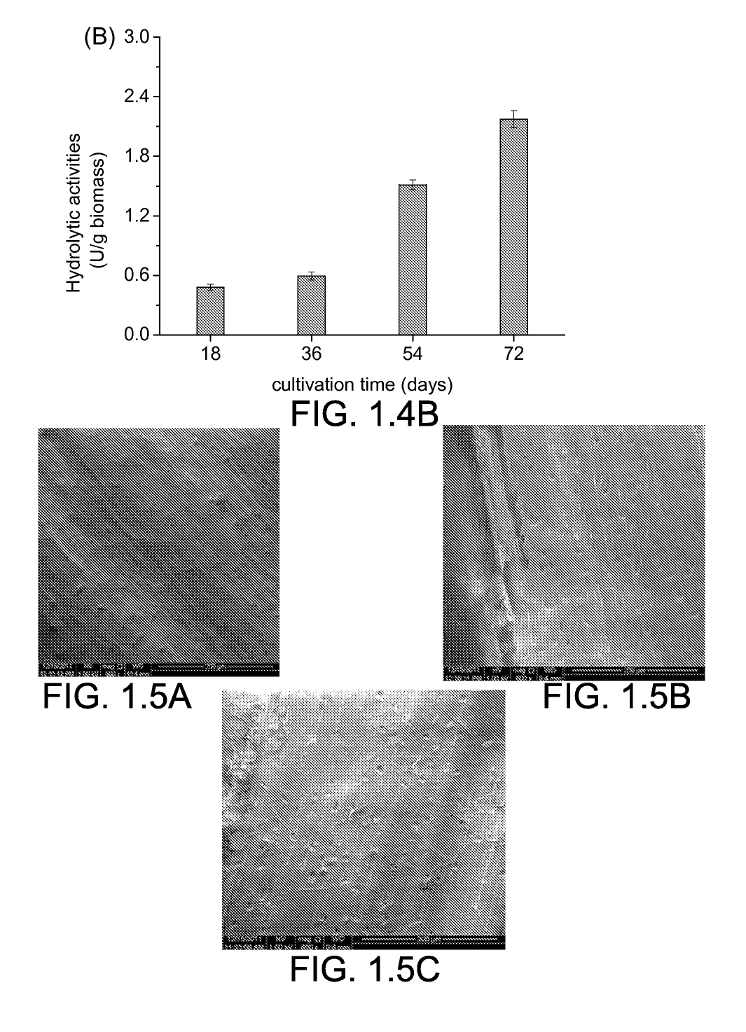

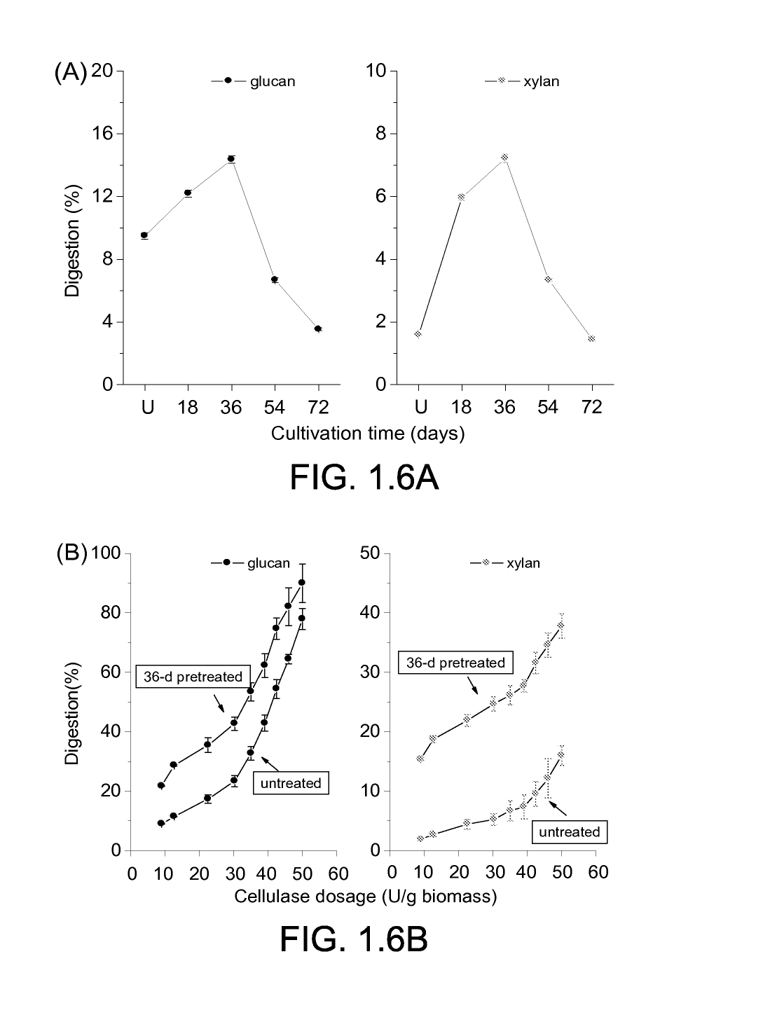

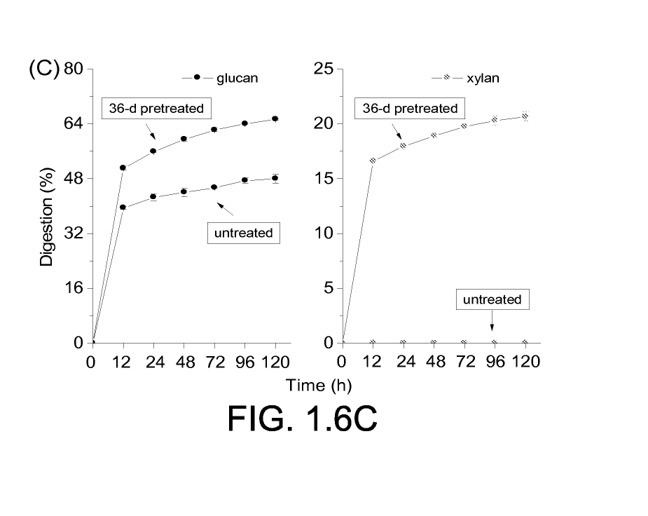

The chemicals used in this study (including standard sugars for HPLC) were all purchased from Sigma unless stated otherwise. Cellulase from Trichoderma viride (Lot# 110M1456V, Japan) and cellulysin cellulase from Trichoderma viride (Cat# 219466, Japan) were purchased from Sigma. Filter paper, NO.1 quality for determination of cellulase activity, was purchased from Whatman™ (W & R Balston Limited, England). 4-Nitrophenyl β-D-glucopyranoside (p-NPG, N1627) and 2,6-dimethoxyphenol (DMP, D135550) was obtained from Sigma as a cromogenic substrate for β-glucosidase and laccase activity determination. The switchgrass biomass (PI 422000) was harvested from the field at the University of Georgia, Griffin Campus in 2010, air dried and stored at room temperature prior to use. Syringeless filter device (Mini-UniPrep™, 0.45 μm Pore Size) from Whatman™ (GE Healthcare UK Limited.) was used to prepare samples for HPLC. The general procedure of this study is illustrated in FIG. 1.1.

### Fungal Strain for Pretreatment of Switchgrass Biomass

The white-rot fungus Pycnoporus sp. SYBC-L3 (18S rRNA sequence was deposited in GenBank with accession number GU182936) was used in this study for biological pretreatment of switchgrass and simultaneous enzymes production. The strain was identified in our previous study as an effective laccase producer (Liu et al., 2012), and its culture stock was stored at The Key Laboratory of Industrial Biotechnology, Ministry of Education, School of Biotechnology, Jiangnan University, (Wuxi, China). The fungus was maintained on potato dextrose agar slants at 4° C. and subcultured on plates at intervals of every two weeks.

### Fungal Pretreatment of Switchgrass Biomass

Solid state fermentation was used for fungal pretreatment of switchgrass biomass in this study. Air-dried switchgrass biomass was cut into small pieces (1.5 to 2 cm long) and five grams of the biomass were transferred into a 200 mL flask supplemented with 15 g distilled sterile water (pH 7.0). Each flask was autoclaved at 121° C. for 30 min and cooled down to room temperature prior to inoculation. Then five disks with a diameter of 0.5 cm cut from the margin of fungal mycelia on potato dextrose agars were transferred into each flask. Fungal growth was carried out under static condition in the flasks at 30° C. for various periods of time. The cultivation was terminated at 18-d, 36-d, 54-d or 72-d, respectively, for crude enzyme extraction, determination of ligninolytic and hydrolytic activities, compositional analysis and subsequent enzymatic hydrolysis. Each fungal pretreatment was performed with two replicates.

### Enzyme Extraction and Activity Determination

After each period of solid state fermentation, 100 mL buffer (0.1 M citrate phosphate, pH 4.8) was added into the flask to fully soak the switchgrass for 24 h at room temperature before filtration. The filtered crude extract was used for determination of enzymatic activity. The pretreated switchgrass biomass separated through filtration was oven dried to constant weight (105° C. for 24 h) and used for chemical composition analysis and subsequent enzymatic hydrolysis.

For enzyme activity determination, all assays were performed in triplicates using a UV-vis spectrophotometer (DU®640B, Beckman, USA) at room temperature unless stated otherwise. LiP activity was determined by oxidation of veratryl alcohol at 300 nm (ε=9,300 M⁻¹ cm⁻¹) (Tien and Kirk, 1988).The reaction mixture contained 400 μl citrate phosphate buffer (0.1 M, pH 3.0), 400 μl sample, and 200 μl veratryl alcohol solution (2 mM). The reaction was initiated by addition of 0.1 mL H₂O₂ (4 mM). MnP activity was determined by oxidation of MnSO4 at 270 nm (ε=11,590 M⁻¹ cm⁻¹) (Boer et al., 2004). Four mL of a reaction mixture contained 3.7 mL citrate phosphate buffer (0.1 M, pH 4.5), 0.1 mL substrate MnSO₄ (10 mM), and 0.1 mL sample, and the reaction was initiated by adding 0.1 mLH₂O₂ (4 mM). Laccase activity was determined by oxidation of DMP at 470 nm (ε=46,900 M⁻¹ cm⁻¹). Three mL of a reaction mixture contained 2.4 mL of 0.1 M citrate phosphate buffer (pH 3.5), 0.5 mL substrate DMP (10 mM), and 0.1 mL sample. One unit of enzyme activity (laccase, LiP, and MnP) was defined as the amount of enzyme to produce 1 mol product per min at room temperature. The activity of each enzyme was recorded as U/mL and then calculated by multiplying the total volume of the crude extract and expressed as U/g biomass of untreated switchgrass (Wan and Li, 2010).

Cellulase activity was determined by the amount of released glucose under the conditions of pH 4.8 (citrate phosphate buffer) and 50° C. according to Laboratory Analytical Procedure (LAP) by National Renewable Energy Laboratory (NREL). One unit is defined as the release of 2.0 mg glucose from 50 mg filter paper in 60 min (NREL/TP-500-42628). Xylanase activity was determined by the amount of released xylose under the conditions of pH 4.8 (citrate phosphate buffer) and 30° C. One unit is defined as the release 1 μmole of xylose from xylan per min (Wan and Li, 2010). Released sugars were measured by HPLC. β-glucosidase activity was determined based on the method described by Turner et al (2002) with some modifications. Specifically, the reaction (2.4 ml citrate phosphate buffer/pH 4,8, 0.1 M; 0.1 ml sample solution; 0.5 ml p-NPG/10 mM) was performed at 50° C. for 10 min and then stopped by adding 1 ml Na₂CO₃/0.5 M. The absorbance was recorded at 405 nm (ε=18,300 mM cm⁻¹), with one unit of activity defined as the amount of enzyme that liberates 1 μmol of p-nitrophenol per minute.

### Scanning Electron Microscope Examination

Some fungal pretreated and untreated switchgrass were examined using a FEI Inspect F50 FEG scanning electron microscope (SEM) at an accelerating voltage of 1.00 kV.

### Chemical Composition of Switchgrass Biomass

Cellulose, hemicellulose, lignin, and ash content were analyzed according to methods described by National Renewable Energy Laboratory (NREL/TP-500-42618). After acid hydrolysis, released sugars were determined using HPLC and calculated for cellulose and hemicellulose. Acid-insoluble lignin and acid-soluble lignin were measured by gravity and UV-Vis spectrophotometer (DU®640B, Beckman, USA), respectively.

### Enzymatic Hydrolysis of Switchgrass Biomass

Enzymatic hydrolysis was carried out according to the methods described by National Renewable Energy Laboratory (NREL/TP-500-42629) with some modifications. 0.1 g of biomass on a dry mass basis, 5.0 mL of 0.1 M citrate phosphate buffer (pH 4.8), 400 μg tetracycline and 300 μg cycloheximide were added to each 20 mL glass scintillation vial. Then enzyme solutions (containing cellulase from Trichoderma viride at different FPU activities) or the sample were added to bring the final volume to 10 mL for each vial. The vials were tightly capped and incubated at 50° C. with constant shaking for 5-d to hydrolyze the cellulose. Finally, liquid samples from each vial were filtered using syringeless filter vials and analyzed by HPLC for sugar content including glucose, xylose, galactose, arabinose, and mannose.

### Sugar quantification by HPLC Analysis

The concentration of released monomeric sugars (including glucose, xylose, mannose, galactose, and arabinose) was determined using HPLC analysis according to the standard methods (NREL/TP-500-42618). Each sample was neutralized to around pH 7.0 using an appropriate amount of sodium bicarbonate and then filtered through a 0.45 PVDF filter membrane prior to injection on an Agilent 1100 Liquid chromatography with a refractive index detector. Ten μL sample solution was injected and run at a flow rate of 0.65 mL/min. Sugar standards from Sigma were measured, dissolved and diluted in water to the following concentration (mg/mL): 0.1, 0.2, 0.5, 1.0, 2.0, and 4.0. Sugar concentrations (mg/mL) generated by HPLC from various samples was used to calculate percent digestion of cellulose and hemicellulose.

Enzymatic Hydrolysis Curve of Pretreated and Untreated Switchgrass Biomass

To compare released monomeric sugars during enzymatic hydrolysis of the untreated and pretreated switchgrass biomass, hydrolysates were sampled at 12, 24, 48, 72, 96, and 120 h, and the amounts of released sugars were calculated and determined from the results of HPLC analysis, accordingly.

### Results and Discussion

### Fungal Growth and Switchgrass Biomass Loss

Air-dried switchgrass biomass contained about 10% moisture, as calculated from the oven dry weight (ODW) (about 4.5 g after drying 5 g of untreated sample) in FIG. 1.2. After the initial inoculation of the substrate (5 g switchgrass in 15 g water), the fungus showed no significant growth for the first 2-d of cultivation, indicating not much nutrition was available for the fungus. By day 4, however, the fungus was growing well and at day 10, it had completely covered the substrate, suggesting that the fungus could degrade switchgrass and take up nutrients from the biomass for its growth and metabolism (FIG. 1.1C). Along with the rapid growth of fungus during cultivation, a significant decrease in biomass ODW was observed. The ODW decreased to 3.8 g, 3.2 g, 2.4 g and 1.9 g at the 18-d, 36-d, 54-d and 72-d cultivation, respectively (FIG. 1.2). The corresponding biomass loss was about 10%, 30%, 50%, 58% for various periods of cultivation time, respectively, which is higher than the results from fungal pretreated corn stover by Ceriporiopsis subvermispora (Wan and Li, 2010). This may be explained by two reasons: the use of a different fungus which may have an increased ability to break down biomass and the use of a different substrate with a different biomass structure.

### Chemical Composition of Fungal Pretreated and Untreated Switchgrass Biomass

Chemical compositions of fungal pretreated and untreated switchgrass biomass are shown in FIG. 1.3. By ODW, the switchgrass biomass was composed of 33% glucan, 18% xylan, 6% acid soluable lignin, 17% acid insoluable lignin, 4% ash and 22% other compounds. This is generally consistent with the previously published results, 30.6-33.6% glucan and 10.4-17.2% acid insoluble lignin (AIL) varying with ecotypes, harvesting time, and cultivation locations (Bals et al., 2010). However, the chemical composition was changed after fungal pretreatment according to the length of cultivation. As shown in FIG. 1.3A, the content of structural sugars (mainly glucan and xylan) increased slightly at cultivation time of 18-d and then decreased gradually as the cultivation time progressed. The acid soluble lignin (ASL) content also increased until at 54-d and then dropped down to the level of untreated at 72-d (FIG. 1.3B). The most dramatic changes were in acid insoluble lignin (AIL) and ash content, as shown in FIG. 1.3C and D. A significant decrease in AIL content after fungal pretreatment was observed at 18-d and 36-d, to 14% and 11% of ODW, respectively, marking an 18% or 35% reduction. AIL content, however, increased to the untreated level at 54-d and then increased still more by 72-d, reaching 16% and 19% of ODW, respectively. Unlike the content changes of structural sugars, ASL and AIL, the percent ash in pretreated switchgrass biomass increased steadily from 4% of untreated ODW to 10% by 72-d of fungal cultivation (FIG. 1.3C). The fungal pretreatment appears to be more effective at lignin removal (especially for a 36-d pretreatment) than the AFEX pretreatment method (which is incapable of removing lignin from biomass) (Bals et al., 2010). Similar results for lignin reduction (41%) were also obtained when Pleurotus ostreatus was employed on the pretreatment of corn stover by Taniguchi et al (2005).

### Ligninolytic and Hydrolytic Enzyme Production During the Fungal Cultivation

During the solid state fermentation of the fungus on switchgrass biomass, ligninolytic and hydrolytic enzymes activities were detected in the crude extract. The activities from three ligninolytic enzymes (LiP, MnP and laccase) were all detected, shown in FIG. 1.4A as gray, green, and blue bars, respectively. Laccase had the highest activity followed by MnP and LiP. All three ligninolytic enzyme activities increased with the cultivation time and reached their peak activity of 8.8 U/g, 2.1 U/g and 1.6 U/g at 54-d, respectively. When the cultivation time extended beyond 54-d, a decrease of ligninolytic activity was observed. As for the hydrolytic enzymes (FIG. 1.4B), cellulase activity using filter paper method was not found for any of the cultivation times. Even though it has been found in a species of the same genus, Pycnoporus sanguineus (UEC-2050 strain), when growing on malt extract (Esposito et al., 1993), xylanase was not detected in this study. This may be attributed to differences in the growth medium used. However, β-glucosidase, which degrades cellubiose into glucose, was observed in the crude extract. An approximate peak activity of 2.2 U/g for β-glucosidase was measured after 72-d cultivation. The secretive ability of this hydrolytic enzyme by the genus Pycnoporus was also demonstrated in another study (De Almeida Siqueira et al., 1997). Among all the ligninolytic and hydrolytic enzymes, laccase was the predominant extracellular enzyme secreted by Pycnoporus sp. SYBC-L3, which was in accordance with another study using Pycnoporus sanguineus (UEC-2050 strain) (Esposito et al., 1993). The laccase production by Pycnoporus sp. SYBC-L3 (8.8 U/g) was slightly higher than that of Ceriporiopsis subvermispora (3.6 U/g) (Wan and Li, 2010). It is also higher than that produced by Trametes hirsuta yj9 (0.8 U/g), showing the wide range of enzyme secretion variation among different fungal strains (Sun et al., 2011).

The biomass loss could be attributed to the fungal growth and the production of extracellular enzymes as co-products. A similar result was also found by Yang et al (2011). Owing to considerable biomass loss and energy cost for bioethanol production, production of valuable co-products may be an alternative for reducing bioethanol cost (Cardona and Sanchez, 2007). In our study, large quantity of extracellular enzymes (mainly laccase) was generated during the fungal pretreatment of switchgrass biomass. Laccase, a multicopper oxidase, has a great potential for use in industrial settings (Rodriguez Couto and Toca Herrera, 2006). In addition to testing for enzyme activities in the crude extract, sugars, including cellobiose, glucose, xylose, galactose, arabinose, and fructose were also measured by HPLC. No sugars were detected in the crude extract, indicating that biological pretreatment using solid state fermentation did not release any monomeric sugars from the polysaccharides in the biomass or the lost biomass was degraded and subsequently metabolized by the fungus.

### Scanning Electron Microscopy of Pretreated and Untreated Switchgrass Biomass

After fungal pretreatment, the integrity of switchgrass stem surface was partially destroyed, forming various pits or holes on the surface which was presumably decomposed or digested by the fungus (FIG. 1.5). Similar SEM pictures regarding porosity on the pretreated surface of corn stover were also demonstrated by a recent study involving Trametes hirsuta yj9 (Sun et al., 2011). The porosity on switchgrass biomass was greatly increased and the surface area that can be exposed to enzymes was thus enlarged. The fungal pretreated switchgrass powder looks more reddish than the untreated one (FIG. 1.1F). Some work on genetic modification of switchgrass has been reported on altering the cellulose/lignin ratio for a better cellulosic ethanol production (Fu et al., 2011). However, because lignin cannot be fully removed from biomass, disruption of lignocellulosic biomass structure prior to enzymatic hydrolysis was still needed. An increase of porosity in pretreated biomass can greatly contribute to a more effective enzymatic hydrolysis (Alvira et al., 2010).

### Enzymatic Hydrolysis of Fungal Pretreated and Untreated Switchgrass Biomass from Various Periods of Cultivation Time

To evaluate the pretreatment effect on switchgrass biomass by the fungus Pycnoporus sp. SYBC-L3, enzymatic hydrolysis of untreated and fungal pretreated switchgrass biomass for various periods of cultivation time was investigated and the results were shown in FIG. 1.6A. For the untreated switchgrass biomass, only 9.5% and 1.6% digestion were obtained for glucan and xylan, respectively. Fungal pretreatment for various time resulted in different effect on digestion of glucan and xylan. Positive effect was found for cultivation time of 18-d and 36-d with 12.2% and 14.4% digestion for glucan and 6.0% and 7.2% digestion for xylan, respectively. A decrease in sugar content resulted when the fungal pretreatment cultivation time was over 54-d. One explanation for this may be the varying lignin content in the biomass. A higher percent of lignin might inhibit enzyme activity and impede accessibility to substrates and thus decrease enzymatic hydrolysis efficiency (Bats et al., 2010). Ash content may also influence the subsequent enzymatic hydrolysis. For biological pretreatment of biomass involving fermentation by fungi, the period of cultivation time has a great influence on biomass digestibility and should be precisely controlled in terms of cellulose consumption and structural alteration. In this study, an approximately of 7% xylan digestion were observed in spite of no xylanase supplementation. This might be due to some xylanase activity that was contained in the cellulase enzyme that we used.

### Enzyme Dosage Effect on Enzymatic Hydrolysis of 36-d Pretreated and Untreated Switchgrass Biomass

FIG. 1.6B. shows the effect of enzyme dosage on enzymatic hydrolysis of switchgrass biomass. Without pretreatment, a higher digestibility of switchgrass was still obtained under conditions of higher enzyme dosage. Fungal pretreatment, however, apparently boosted enzymatic hydrolysis compared with untreated one at the same dosage. With high cellulase dosage, around 90% and 75% digestion of glucan were achieved for pretreated and untreated switchgrass, respectively, but xylan could not be hydrolyzed at a high percentage because of low xylanase activity in cellulase enzyme (Dien et al., 2008). Because xylose can also be utilized for ethanol production by certain microorganisms, supplementation of xylanase can be used for releasing a higher amount of total sugars (Wan and Li, 2010). Our results showed that a higher cellulase dosage could increase glucose and xylose yields but with an increased cost, consistent with a previous report (Li et al., 2007).

### Kinetics of Glucose and Xylose Released During Enzymatic Hydrolysis of 36-d Pretreated and Untreated Switchgrass Biomass

To better understand the time course of sugar releasing and further assess the effect of fungal pretreatment on enzymatic hydrolysis of switchgrass, monomeric sugar releasing profiles of pretreated and untreated switchgrass biomass were collected at a fixed enzyme dosage for various incubation times. Samples were withdrawn at intervals (10, 24, 48, 72, 96, and 120 h) during a 5-d enzymatic hydrolysis. As shown in FIG. 1.6C, fungal pretreated and untreated switchgrass biomass responded differently to enzymatic hydrolysis, although both increased digestion percent along with extended reaction time. More cellulosic biomass was digested into glucose and xylose for the 36-d pretreatment. Xylose released from untreated switchgrass was lower compared with that in FIG. 1.6A, which might be because that different batches of enzymes have been used in FIG. 1.6B and C experiments as indicated in their respective captions. During enzymatic hydrolysis, the highest rate was obtained in the first 12 h hydrolysis of cellulose into glucose for untreated and fungal pretreated switchgrass. Likewise, 36-d pretreated switchgrass rendered the highest release of xylose during the first 12 h hydrolysis. A similar result for glucose and xylose was also reported by Bals et al (2010). Previous studies have found that more xylose was released than glucose at a shorter time while more glucose at a longer time for switchgrass biomass pretreated with diluted H₂SO₄ and SO₂ (Shi et al., 2011). A similar trend for xylose/glucose releasing was also observed for AFEX pretreated switchgrass among different cultivars and harvests except the July harvest of CIR switchgrass biomass (Bals et al., 2010). In this study, our data showed a different result for releasing rate of glucose and xylose, with both having higher rates at shorter time (≦10 h), which might be a result of different pretreatment approaches.

Considering the feasibility of industrial application of biomass pretreatment, the energy input or production cost must be taken into account. Current technologies relating to biomass pretreatment are mostly chemical or physical or physico-chemical combination methods, which usually have great concerns on cost and environmental consequences. Some physical or chemical methods seem to have shorter pretreatment periods (Wang et al., 2011) than the biological method in this study. However, reagents and chemicals involved in pretreatment process may generate new pollutants to environment, and the reaction temperature can be as high as 450° C. (Wang et al., 2011). The bioenergy produced from biomass can be largely offset by high temperature input for pretreatment, which reduced the feasibility. An efficient pretreatment method called ionic-liquid pretreatment offered a better result for releasing sugars compared to diluted-acid pretreatment (Li et al., 2010). However, the cost and subsequent management of ionic liquid must also be taken into consideration. Although steam treatment of biomass has been viewed as one of the most effective methods, its energy input (121° C. for 96 h) is still a considerable cost (Li et al., 2007).

Biological pretreatment involving fungus cultivation, like the one we studied, has numerous advantages in terms of sustainability, recyclability, compatibility and environmental protection. The temperature required for the process is much lower than most physicochemical approaches, thus considerably reducing energy input. The enzyme co-products measured in this study can be employed for treatment of environmental pollutions and other important usages, thus offsetting the cost of fuel production. In particular, we in a companion study demonstrated the use of the crude extract from the fungal cultivation, containing the enzyme co-products, to effectively alleviate soil water repellency, a common problem occurring worldwide that can cause severe reduction in crop productivity and turf quality (Liu et al., 2012). Using our data as an estimation basis, one ton of switchgrass ($80/ton) can potentially produce up to 6,000,000 U laccase. Sigma-Aldrich sells 10,000 U laccase (from Trametes versicolor, Sigma) for $89, indicating this could be a cheaper alternative source of this enzyme. The production costs can be further minimized by optimizing conditions for the entire process, including fermentation, enzyme extraction, hydrolysis, and ethanol production.

### Conclusions

Fungal pretreatment of switchgrass resulted in chemical composition changes and improved saccharification efficiency. The fungus Pycnoporus sp. SYBC-L3 has the capability to selectively decompose lignin over cellulose and hemicellulose in switchgrass. The disrupted lignocellulosic structures and the added porosity on pretreated switchgrass may be the primary causes for improved hydrolysis efficiency. Co-products, mainly laccase and β-glucosidase, were produced which can help to reduce the bioethanol production cost. Our results provide a good model for biomass pretreatment, which can be potentially cost-saving and environmentally friendly and should be explored on other bioenergy feedstocks as well.

### References, Each of Which is Incorporated Herein by Reference

1 Alvira, P., Tomas-Pejó, E., Ballesteros, M., Negro, M. J., 2010. Pretreatment technologies for an efficient bioethanol production process based on enzymatic hydrolysis: A review. Bioresource Technol. 101, 4851-4861.

2 Balan, V., da Costa Sousa, L., Chundawat, S., Vismeh, R., Jones, A., Dale, B., 2008. Mushroom spent straw: a potential substrate for an ethanol-based biorefinery. J. Ind. Microbiol. Biotech. 35, 293-301.

3 Bals, B., Rogers, C., Jin, M., Balan, V., Dale, B., 2010. Evaluation of ammonia fibre expansion (AFEX) pretreatment for enzymatic hydrolysis of switchgrass harvested in different seasons and locations. Biotechnol. Biofuels. 3, 1-11.

4 Boer, C. G., Obici, L., Souza, C. G. M.d., Peralta, R. M., 2004. Decolorization of synthetic dyes by solid state cultures of Lentinula (Lentinus) edodes producing manganese peroxidase as the main ligninolytic enzyme. Bioresource Technol. 94, 107-112.

5 Cardona, C. A., Sanchez, Ó. J., 2007. Fuel ethanol production: Process design trends and integration opportunities. Bioresource Technol. 98, 2415-2457.

6 De Almeida Siqueira, E. M., Mizuta, K., Giglio, J. R., 1997. Pycnoporus sanguinous: a novel source of α-amylase. Mycol. Res. 101, 188-190.

7 Dien, B. S., Ximenes, E. A., O'Bryan, P. J., Moniruzzaman, M., Li, X.-L., Balan, V., Dale, B., Cotta, M. A., 2008. Enzyme characterization for hydrolysis of AFEX and liquid hot-water pretreated distillers' grains and their conversion to ethanol. Bioresource Technol. 99, 5216-5225.

8 Eggert, C., Temp, U., Eriksson, K. E., 1997. Laccase is essential for lignin degradation by the white-rot fungus Pycnoporus cinnabarinus. FEBS Lett. 407, 89-92.

9 Esposito, E., Innocentini-Mei, L. H., Ferraz, A., Canhos, V. P., Duran, N., 1993. Phenoloxidases and hydrolases from Pycnoporus sanguineus (UEC-2050 strain): applications. J. Biotechnol. 29, 219-228.

10 Esteghlalian, A., Hashimoto, A. G., Fenske, J. J., Penner, M. H., 1997. Modeling and optimization of the dilute-sulfuric-acid pretreatment of corn stover, poplar and switchgrass. Bioresource Technol. 59, 129-136.

11 Ewanick, S., Bura, R., 2011. The effect of biomass moisture content on bioethanol yields from steam pretreated switchgrass and sugarcane bagasse. Bioresource Technol. 102, 2651-2658.

12 Faga, B. A., Wilkins, M. R., Banat, I. M., 2010. Ethanol production through simultaneous saccharification and fermentation of switchgrass using Saccharomyces cerevisiae D5A and thermotolerant Kluyveromyces marxianus IMB strains. Bioresource Technol. 101, 2273-2279.

13 Fu, C., Mielenz, J. R., Xiao, X., Ge, Y., Hamilton, C. Y., Rodriguez, M., Chen, F., Foston, M., Ragauskas, A., Bouton, J., Dixon, R. A., Wang, Z.-Y., 2011. Genetic manipulation of lignin reduces recalcitrance and improves ethanol production from switchgrass. P. Natl. Acad. Sci. 108, 3803-3808.

14 George, N., Yang, Y., Wang, Z., Sharma-Shivappa, R., Tungate, K., 2010. Suitability of canola residue for cellulosic ethanol production. Energ. Fuel. 24, 4454-4458.

15 Hu, Z., Wen, Z., 2008. Enhancing enzymatic digestibility of switchgrass by microwave-assisted alkali pretreatment. Biochem. Eng. J. 38, 369-378.

16 Li, A., Antizar-Ladislao, B., Khraisheh, M., 2007. Bioconversion of municipal solid waste to glucose for bio-ethanol production. Bioproc. Biosyst. Eng. 30, 189-196.

17 Li, A. D., Khraisheh, M., 2010. Bioenergy II: Bio-ethanol from municipal solid waste (MSW): The role of biomass properties and structures during the ethanol conversion process. Int. J. Chem. React. Eng. 8, A 85.

18 Li, C., Knierim, B., Manisseri, C., Arora, R., Scheller, H. V., Auer, M., Vogel, K. P., Simmons, B. A., Singh, S., 2010. Comparison of dilute acid and ionic liquid pretreatment of switchgrass: Biomass recalcitrance, delignification and enzymatic saccharification. Bioresource Technol. 101, 4900-4906.

19 Liu, J., Cai, Y., Liao, X., Huang, Q., Hao, Z., Hu, M., Zhang, D., 2012. Simultaneous laccase production and color removal by culturing fungus Pycnoporus sp. SYBC-L3 in a textile wastewater effluent supplemented with a lignocellulosic waste Phragmites australis. B. Environ. Contam. Tox. DOI: 10.1007/s00128-00012-00678-00128.

20 Liu, J., Zeng, L., Carrow, R. N., Raymer, P. L., Huang, Q., 2012. A novel approach for alleviation of soil water repellency using a crude enzyme extract from fungal pretreatment of switchgrass. Bioresource Technol. submitted,

21 Matsushika, A., Inoue, H., Murakami, K., Takimura, O., Sawayama, S., 2009. Bioethanol production performance of five recombinant strains of laboratory and industrial xylose-fermenting Saccharomyces cerevisiae. Bioresource Technol. 100, 2392-2398.

22 McLaughlin, S. B., Walsh, M. E., 1998. Evaluating environmental consequences of producing herbaceous crops for bioenergy. Biomass. Bioenerg. 14, 317-324.

23 Pallapolu, V. R., Lee, Y. Y., Garlock, R. J., Balan, V., Dale, B. E., Kim, Y., Mosier, N. S., Ladisch, M. R., Falls, M., Holtzapple, M. T., Sierra-Ramirez, R., Shi, J., Ebrik, M. A., Redmond, T., Yang, B., Wyman, C. E., Donohoe, B. S., Vinzant, T. B., Elander, R. T., Hames, B., Thomas, S., Warner, R. E., 2011. Effects of enzyme loading and β-glucosidase supplementation on enzymatic hydrolysis of switchgrass processed by leading pretreatment technologies. Bioresource Technol. 102, 11115-11120.

24 Rodriguez Couto, S., Toca Herrera, J. L., 2006. Industrial and biotechnological applications of laccases: A review. Biotechnol. Adv. 24, 500-513.

25 Shi, J., Ebrik, M. A., Wyman, C. E., 2011. Sugar yields from dilute sulfuric acid and sulfur dioxide pretreatments and subsequent enzymatic hydrolysis of switchgrass. Bioresource Technol. 102, 8930-8938.

26 Sun, F.-h., Li, J., Yuan, Y.-x., Yan, Z.-y., Liu, X.-f., 2011. Effect of biological pretreatment with Trametes hirsuta yj9 on enzymatic hydrolysis of corn stover. Int. Biodeter. Biodegr. 65, 931-938.

27 Taniguchi, M., Suzuki, H., Watanabe, D., Sakai, K., Hoshino, K., Tanaka, T., 2005. Evaluation of pretreatment with Pleurotus ostreatus for enzymatic hydrolysis of rice straw. J. Biosci. Bioeng. 100, 637-643.

28 Taniguchi, M., Takahashi, D., Watanabe, D., Sakai, K., Hoshino, K., Kouya, T., Tanaka, T., 2010. Evaluation of fungal pretreatments for enzymatic saccharification of rice straw. J. Chem. Eng. Jpn. 43, 401-405.

29 Tien, M., Kirk, T.-K., 1988. Lignin peroxidase of Phanerochaete chrysosporium. Method. Enzymol. 161, 238-249.

30 Turner, B. L., Hopkins, D. W., Haygarth, P. M., Ostle, N., 2002. β-Glucosidase activity in pasture soils. Appl. Soil. Ecol. 20, 157-162.

31 Wan, C., Li, Y., 2010. Microbial delignification of corn stover by Ceriporiopsis subvermispora for improving cellulose digestibility. Enzyme. Microb. Technol. 47, 31-36.

32 Wang, H., Srinivasan, R., Yu, F., Steele, P., Li, Q., Mitchell, B., 2011. Effect of acid, alkali, and steam explosion pretreatments on characteristics of bio-oil produced from pinewood. Energ. Fuel. 25, 3758-3764.

33 Yang, H., Wu, H., Wang, X., Cui, Z., Li, Y., 2011. Selection and characteristics of a switchgrass-colonizing microbial community to produce extracellular cellulases and xylanases. Bioresource Technol. 102, 3546-3550.

34 Yang, Y., Sharma-Shivappa, R., Burns, J. C., Cheng, J. J., 2009. Dilute acid pretreatment of oven-dried switchgrass germplasms for bioethanol production. Energ. Fuel. 23, 3759-3766.

35 Zeng, Y. L., Yang, X. W., Yu, H. B., Zhang, X. Y., Ma, F. Y., 2011. Comparative studies on thermochemical characterization of corn stover pretreated by White-Rot and Brown-Rot Fungi. J. Agric. Food Chem. 59, 9965-9971.

### Example 2

### Brief Introduction

Lignocellulosic materials are renewable resources for bioethanol production from sugars. Pretreatment of lignocellulosic materials is a necessary element in bioconversion of cellulosic and hemicellulosic sugars to ethanol. Removal of lignin from lignocellulosic biomass was optimized by laccase-mediator system. A 300 mg sample of sweet sorghum and switchgrass in a 50 mL Erlenmeyer flask was subjected to 20 mL reaction mixture of laccase or laccase mediator system (LMS). Activity of laccase enzyme in the reaction mixture was 10 units mL⁻¹ along with varying concentrations of one of the three mediators; HBT (1-hydroxybenzotriazole), violuric acid (5-isonitrosobarbituric acid), and ABTS (2, 2′-azino-bis (3-ethylbenzothiazoline-6-sulphonic acid). The concentration of HBT and ABTS in the reaction mixture was 0, 0.13, 0.25, 0.31, 0.63, 1.25, and 1.88 mM and violuric acid concentration was 0.31, 0.63, 1.25, and 1.88 mM to optimize the mediator concentration for maximum lignin removal. A 5-8% and 5-14% lignin removal was observed sweet sorghum and switchgrass, respectively after 24 h treatment with laccase-ABTS system. A 25.5% lignin removal at 1.88 mM HBT and 24% at 1.25 mM VA after 24 h treatment was observed in sweet sorghum. Similarly, in switchgrass a 28% lignin removal at 0.63 mM VA in combination with laccase was observed after 24 h treatment. A slight loss is structural sugars were observed at treatments with enzymatic treatments. The optimum mediator concentration for maximum removal of lignin changed with mediators and lignocellulosic biomass.

### Introduction

Population growth and industrial development has led to increased energy consumption in the world, which has increased 17-fold over the period of last 100 years (Ayhan, 2007). The conventional energy resources like fossil fuels are limited in quantity and cannot meet the increasing energy demand, besides having a considerable negative environmental impact such as emitting greenhouse gases like carbon dioxide. Use of alternate and renewable energy options like biofuels have several advantages such as lower CO₂ emissions and lesser dependency of non-oil producing nations for crude oil imports, and has thus drawn widespread attention.

Lignocellulosic materials are heterogeneous complexes of cellulose, hemicellulose and lignin. Removal of lignin from plant cell wall opens up the structure of lignocellulosic biomass and leads to opening of the pore-space in the cell wall structure and thus increase cellulose accessibility for further hydrolysis (Conte et al., 2009; Fang et al., 2010; Kaparaju et al., 2009; Kerr, 1975; Matsushita et al., 2009). Lignocellulosic biomass has to undergo a pretreatment for bioconversion of polymeric sugars to monomers and further fermentation to ethanol (Cheng et al., 2008). Pretreatment recognized as a key step in the bioethanol conversion process must improve the availability of sugars (both cellulosic and hemicellulosic) from enzymatic hydrolysis, prevent loss of sugars or carbohydrates, and avoid formation of chemical inhibitors for subsequent hydrolysis and fermentation processes.

The several methods that have been used as pretreatment till date include dilute acid hydrolysis, wet oxidation, and steam explosion. However, commonly known disadvantages are associated with the existing pretreatment methods including loss of carbohydrates (Abatzoglov et al., 1986; Bouchard et al., 1989; Bouchard et al., 1992; Conner et al., 1985), modification of cellulose polymers or oligomers (Abatzoglov et al., 1986; Bouchard et al., 1989; Mok et al., 1992; Qian et al., 2005), and re-polymerization reaction among carbohydrates by products and lignin intermediates (Li et al., 2007; Xiang et al., 2004).

Natural degradation of lignin is carried out in the environment by certain white-rot fungi which solubilize and mineralize lignin with the help of lignolytic enzymes (Kirk et al., 1975; 1976). White-rot fungi preferentially attack lignin more than cellulose or hemicellulose in the wood tissue (Blanchette, 1984).

Laccase is one of the lignolytic enzymes secreted during oxygen dependent degradation of organic material by white-rot fungi (Ten Have and Teunissen, 2001). Low oxidation potential of laccase restricts its ability to oxidize non-phenolic lignin components (Kersten et al., 1990; Ten Have and Teunissen, 2001). However, addition of low molecular weight substances, mediators, increases the substrate range of laccase enzyme to non-phenolic groups, benzyl and alyl alcohols and ethers (Bourbonnais and Paice, 1992; Bourbonnais et al., 1997; Crestini and Argyropoulos, 1998; Fabbrini et al., 2002; Fabbrini et al., 2001) which comprise the major moieties in lignin macromolecule (Fritz-Langhals and Kunath, 1998; Johannes and Majcherczyk, 2000; Potthast et al., 1995).

In laccase mediator system, the oxidized mediator with a higher redox potential than laccase, acts on the substrate to carry outs its oxidation (Cantarella et al., 2003). Oxidation of organic substrates in laccase-mediator system can proceed by two different mechanisms (Cantarella et al., 2003). In case of mediators like ABTS, the oxidation of substrate is carried out by single electron oxidation, whereas for N—OH type mediators like HBT and violuric acid, the oxidation is carried out by abstraction of H atom by a >N—O radical species.

There are several studies indicating that mediators enhance the oxidation of substrate of laccase (Kang et al., 2002; Kim and Nicell, 2006; Tsutsumi et al., 2001). However, little has been done to optimize the use of mediators in lignin removal from lignocellulosic biomass. We hypothesize that the use of lignin-degrading enzyme such as fungal laccases when applied along with mediators can effectively reduce lignin content from lignocellulosic biomass and our study was designed to test two specific hypothesis: 1) degradation of lignin can be enhanced by laccase-mediator system; and 2) Different mediators have different optimum effectiveness in reducing lignin.

### MATERIALS AND METHODS

### Biomass Preparation

The sweet sorghum [Sorghum bicolor L., PI 17077) and switchgrass (Panicum virgatum L.) biomass were obtained from Dr. M. L. Wang's USDA laboratory at The University of Georgia, Griffin Campus. The biomass sample was ground (177-841 μm) and extracted with water and alcohol to remove water-and alcohol-soluble extractives as specified by the protocol developed by National Renewable Energy Laboratory (NREL, 2008a). The extractive-free biomass samples were air-dried to a moisture level less than 10% before treatment application.

### Chemicals

Laccase used in the experiment was obtained from Jiangnan University, China. The enzyme was purified from a laccase producing fungal strain Pycnoporus sp. SYBC-L3 with accession number GU 182936. Three mediators used in the experiment; HBT (1-hydroxybenzotriazole), violuric acid (5-Isonitrosobarbituric acid), and ABTS (2,2′-azino-bis(3-ethylbenzothiazoline-6-sulphonic acid) along with surfactant, Pluronic F-68 (polyoxyethylene-polyoxypropylene polymer,C₃H₆O.C₂H₄O) were purchased from Sigma-Aldrich (Sigma Aldrich Inc., St. Louis, Mo.).

### Laccase Activity Assay

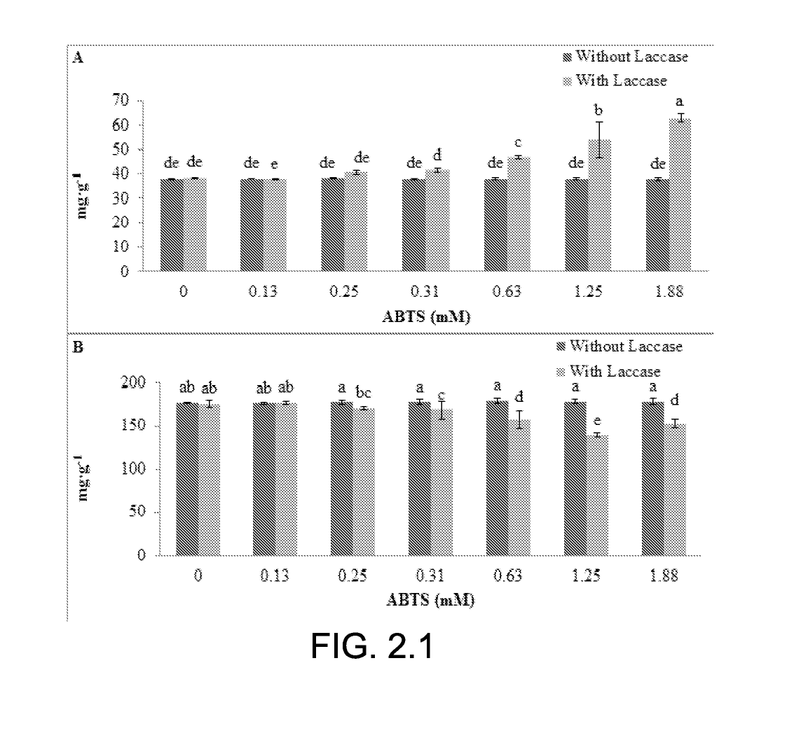

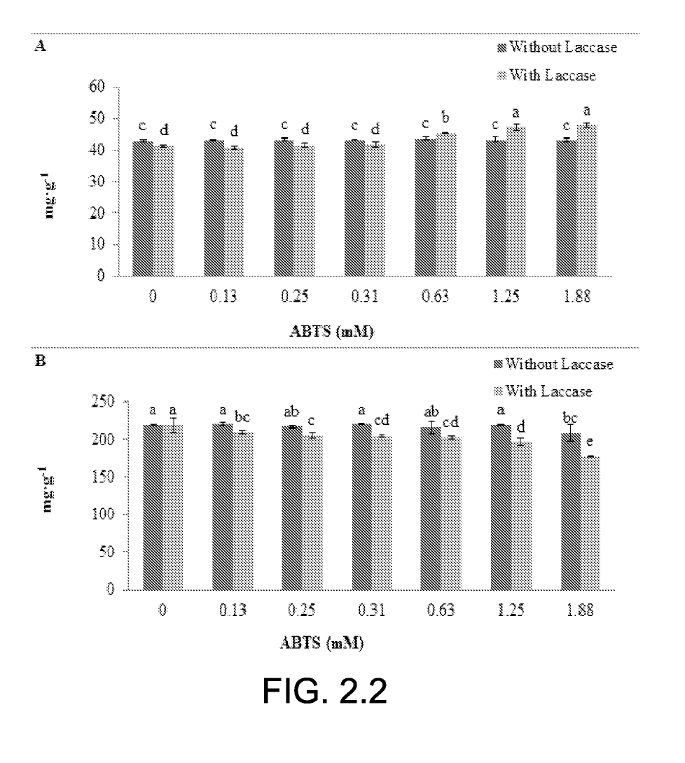

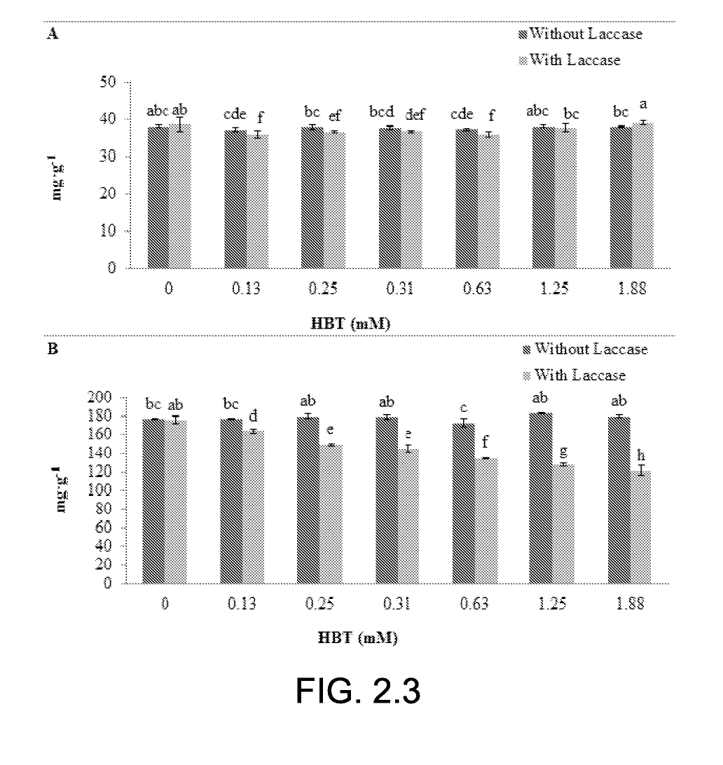

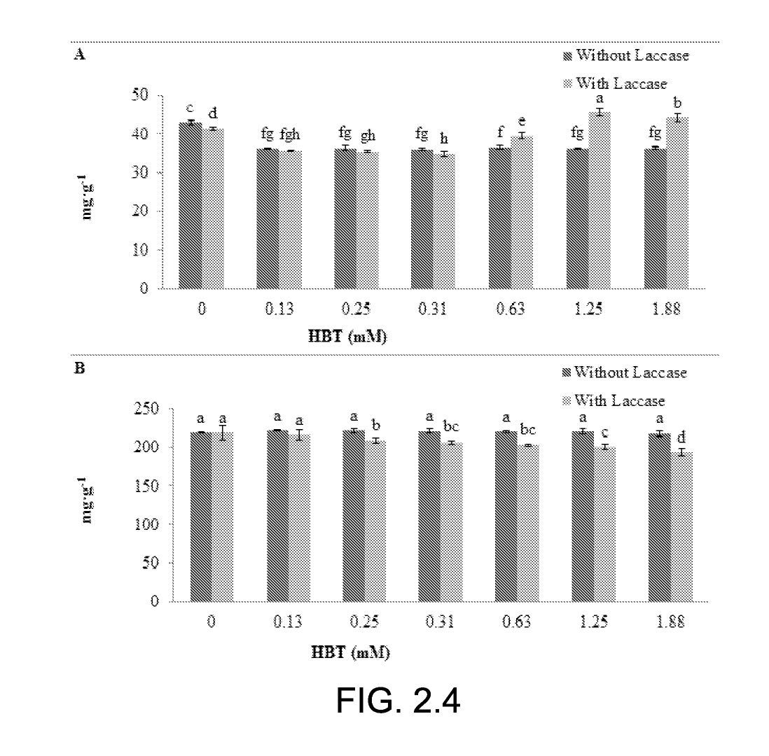

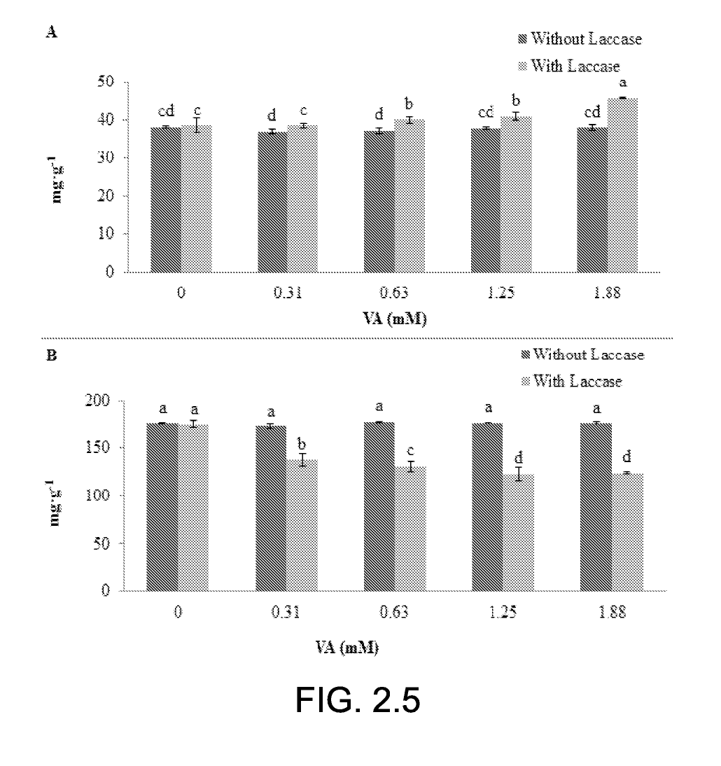

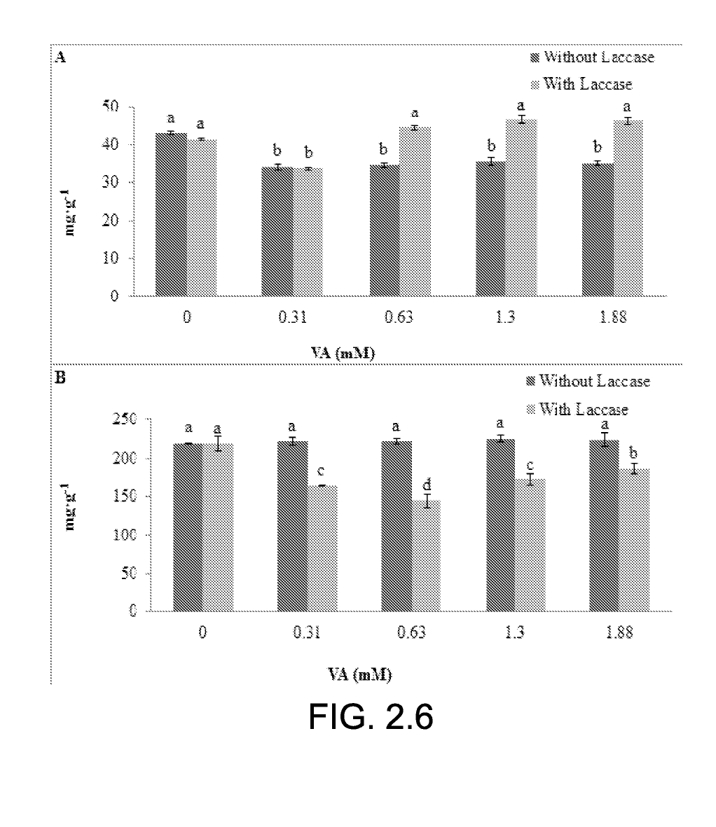

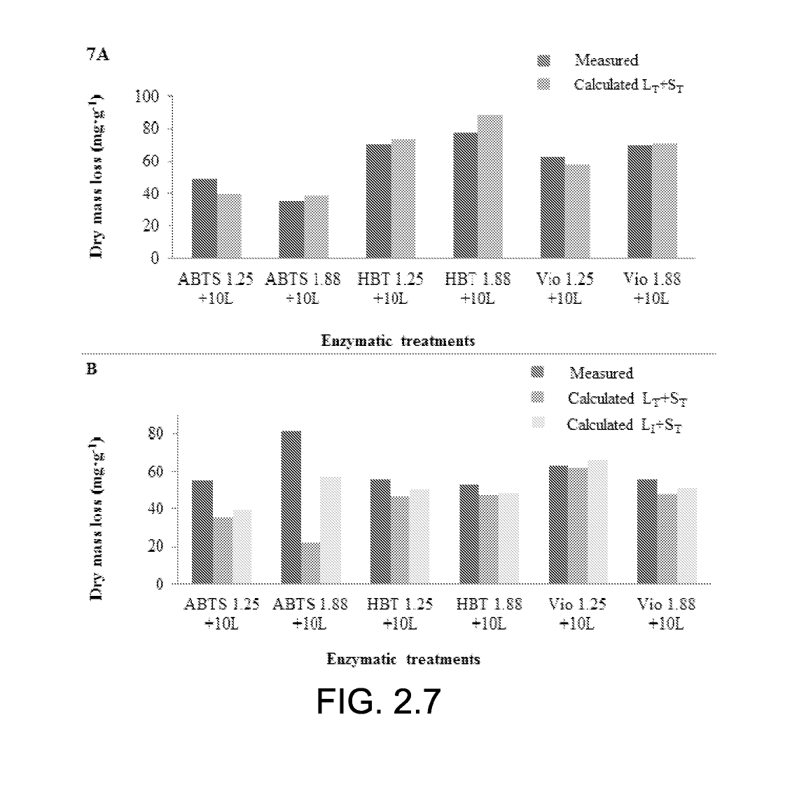

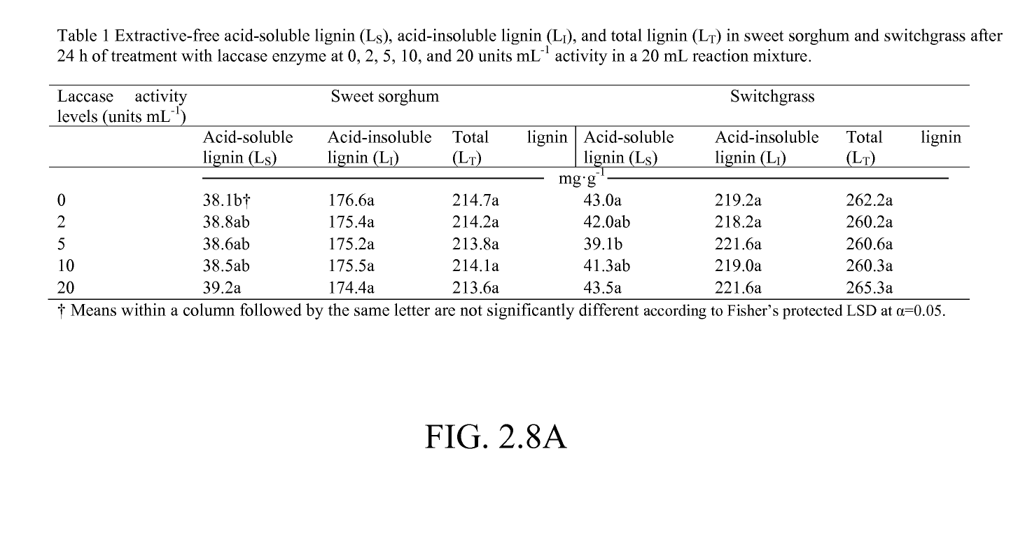

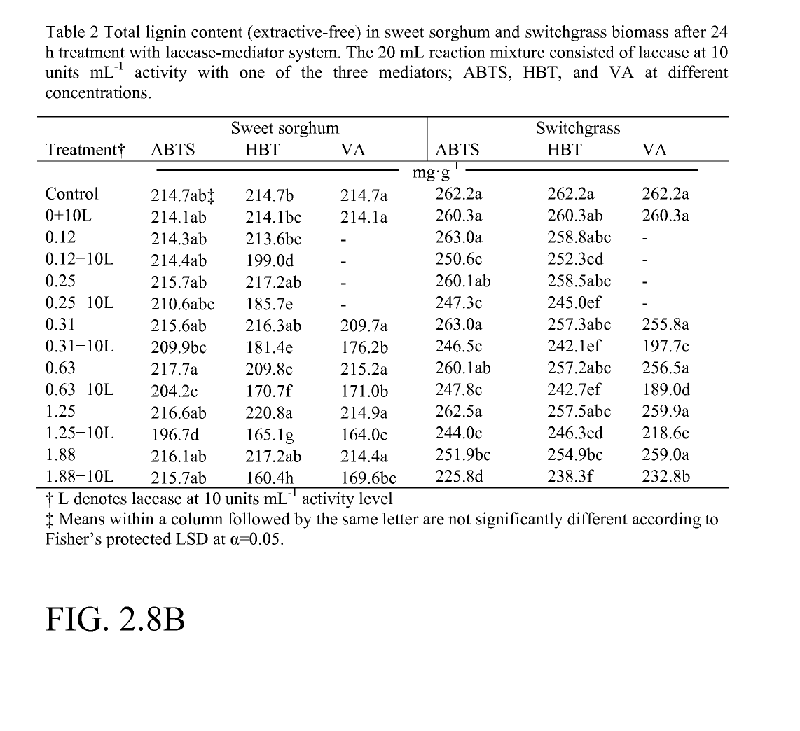

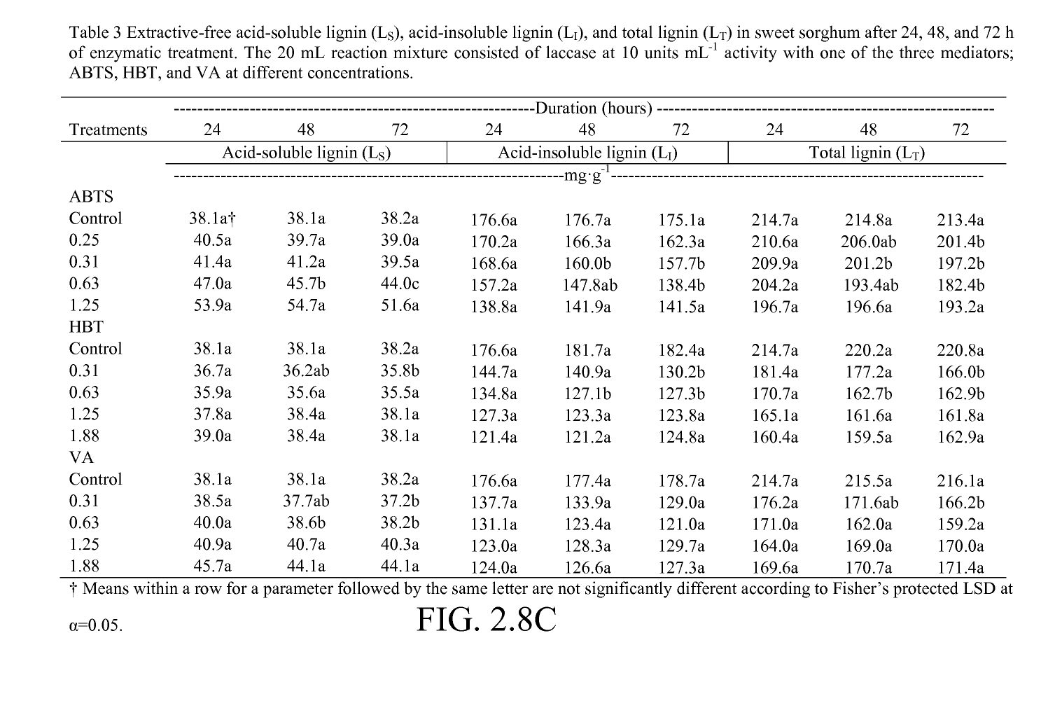

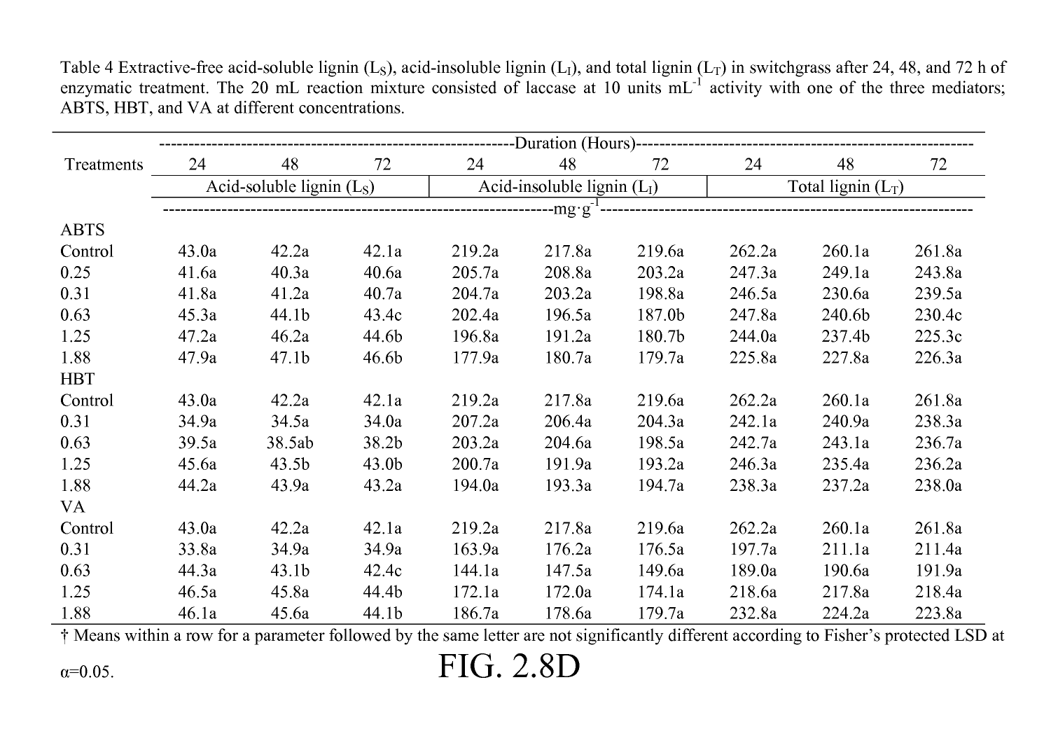

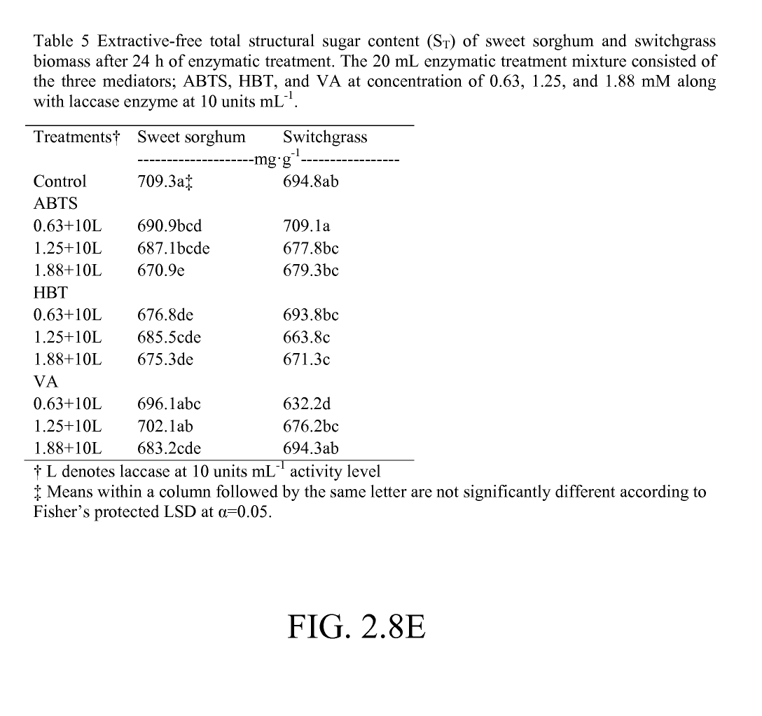

The activity of laccase was quantified using a UV/VIS-spectrophotometer by a colorimetric assay. One activity unit of laccase corresponds to the amount of enzyme that causes an absorbance change at 468 nm at a rate of 1.0 unit min⁻¹ in 3.4 mL of 1 mM 2,6-dimethoxyphenol in citrate-phosphate buffer at a particular pH (Park et al., 1999). Laccase enzyme activity was assayed over a range and was found to be most active at pH 3.0 in the initial laboratory tests conducted to optimize the pH for this enzyme (FIG. 2.1).

### Enzymatic Treatment

The reaction mixture consisted of lignocellulosic biomass, surfactant, laccase, citrate-phosphate buffer at pH 3.0, and mediators. A 300 mg extractive-free biomass sample of sweet sorghum and switchgrass was collected in a 50 mL Erlenmeyer flask and was allowed to stand overnight in a 10 mL solution (3% w/w) of surfactant, Pluronic F-68. Effectiveness of laccase enzyme was observed at activity level of 0, 2, 5, 10 and 20 units mL⁻¹ in the reaction mixture. Laccase mediator system consisted of laccase at 10 units mL⁻¹ along with one of the three mediators. The concentration of HBT and ABTS in the reaction mixture was 0.13, 0.25, 0.31, 0.63, 1.25, and 1.88 mM and violuric acid concentration was 0.31, 0.63, 1.25, and 1.88 mM to examine the effect of mediator concentration on lignin removal. The reaction mixture was put on rotary shaker at 150 rpm at 25° C. Samples were removed from the reaction mixture after 24, 48, and 72 h and were washed three times with 100 mL water.

### Measurements

After enzymatic treatment the samples were dried and weighed to observe dry mass loss. The effectiveness of treatments was determined by measuring acid-soluble lignin (L_{S}) and acid-insoluble lignin (L_{I}) and structural sugars. The lignin and sugar content will be designated as extractive-free lignin and extractive-free structural sugar content due to the use of extractive-free biomass for the experiment. Total lignin (L_{T}) was obtained by addition of acid-soluble and-insoluble lignin contents.

### Extractive-free Lignin Content

Lignin content in the sweet sorghum and switchgrass biomass was determined in a two-step hydrolysis procedure according to the laboratory analytical procedure developed by The National Renewable Energy Laboratory (NREL, 2008b). In the first step, 100 mg of air dried treated biomass samples were hydrolyzed for 60 min with 1 mL of 72% H₂SO₄ at 30° C. in a water bath. In the second step, H₂SO₄ was diluted to 4% and the samples were autoclaved at 121° C. for 1 h. After autoclave the samples were vacuum filtered and the hydrolysis liquid was used for analyzing acid-soluble lignin content and structural sugars. Acid-soluble lignin was determined using this hydrolysis liquid at 240 nm wavelength in a UV/VIS spectrophotometer. The solids remaining after acid hydrolysis were dried in an oven at 100±5° C. for 24 h, weighed, ashed in a muffle furnace at 600±10° C. for 24 h, and weighed again. Weight difference was used to calculate the extractive-free acid-insoluble lignin content.

### Structural Sugars

Structural sugar content for glucose, xylose, arabinose, mannose, and galactose was determined for selected samples from the hydrolysis liquid collected after vacuum filtration in the above step. The hydrolysis liquid was neutralized to a pH range 6.0-8.0 using NaHCO₃ (sodium bicarbonate) and structural sugars were determined using high performance liquid chromatography (HPLC) in an Agilent 1100 HPLC (Aligent Technologies, Waldbronn, Germany) with a binary pump and a refractive index detector. An AMINEX HPX-87P 7.8×300 mm Pb²⁺ carbohydrate analysis column (Bio-Rad, Hercules, Calif.) was used at 85° C. with deionized water as mobile phase at a flow rate of 0.6 mL min⁻¹. These monosaccharide sugars are components of structural polysaccharides, cellulose and hemicellulose. Total sugars were calculated by addition of the sugar contents of these monomers.

### Statistical Analysis

Analysis of variance (ANOVA) was performed to evaluate the main effects of laccase, mediator, and interaction effects of these two factors using general linear model (GLM) (SAS Institute, 1989). Fisher's protected LSD test with α=0.05 was used for determining statistical differences among treatment means following each ANOVA.

### RESULTS

### Lignin Content

### Laccase Application

Treatment of sweet sorghum and switchgrass with different levels of laccase for 24 h had no effect on extractive-free acid-insoluble and total lignin content (Table 1). However, slight but significant differences were observed for L_{S} content (Table 1). A slight increase in L_{S} content (P≦0.05) of sweet sorghum was obtained when reaction mixture consisted of laccase enzyme at 20 units mL⁻¹ whereas in switchgrass a slight reduction in L_{S} content (P≦0.05) was observed in reaction mixture containing 5 units mL⁻¹ laccase activity level as compared to the control (Table 1).

### Laccase-ABTS Mediator System

A significant effect (P≦0.001) of laccase-ABTS system was observed on L_{S}, L_{I} and L_{T} content of sweet sorghum and switchgrass. After 24 h of laccase-ABTS treatment, L_{S} content of sweet sorghum increased by 8.9 (23%), 15.8 (41%), and 24.7 (65%) mg·g⁻¹ over the control with ABTS concentration of 0.63, 1.25, and 1.88 mM, respectively when applied along with laccase (FIG. 2.1A). However, when compared with control, L_{I} content decreased by 8.0 (5%), 19.4 (11%), 37.7 (21%), and 23.7 (13%) mg·g⁻¹ in reaction mixture containing laccase along with ABTS at 0.31, 0.63, 1.25, 1.88 mM, respectively (FIG. 2.1B). A slight reduction of 5 and 8% in total lignin content when compared to control was obtained when ABTS was applied at 0.63 and 1.25 mM along with laccase (Table 2).

Switchgrass biomass treated with laccase-ABTS system showed a slight but significant reduction in L_{S} content up to ABTS concentration of 0.31 mM when applied along with laccase. Extractive-free acid soluble content increased over the control by 2.3, 4.2, and 4.8 mg·g⁻¹ when with ABTS concentration of 0.63, 1.25, and 1.88 mM, respectively, in the reaction mixture containing laccase (FIG. 2.2A). Extractive-free acid-insoluble lignin content of switchgrass biomass was lowered by 9.4, 13.4, 14.4, 16.7, 22.4, and 41.2 mg·g⁻¹ when ABTS concentration was 0.13, 0.25, 0.31, 0.63, 1.25, and 1.88 mM, respectively, in the presence of laccase (FIG. 2.2B). Similarly, L_{T} content in switchgrass biomass decreased in the range of 12.2 (5%) and 36.4 (14%) mg·g⁻¹ in comparison with control for the same concentration of ABTS (Table 2). Reaction mixture containing ABTS at different concentrations without laccase had no effect on acid-soluble and-insoluble lignin content in sweet sorghum and switchgrass (FIGS. 2.1A, 2.1B and FIGS. 2.2A, 2.2B).

Duration of laccase-ABTS system had no effect on Lₛ content in sweet sorghum for ABTS concentration of 0.25, 0.31, and 1.25 mM (Table 3). After 48 and 72 h of treatment at ABTS concentration of 0.63 mM, a reduction in L_{S} content was observed (Table 3). However, a reduction in L_{S} content was observed with increase in treatment duration switchgrass biomass when ABTS concentration was 0.63 mM or higher in laccase-ABTS system (Table 4). No duration effect was observed for L_{I} content in sweet sorghum for ABTS concentrations of 0.25 and 1.25 mM (Table 3). When ABTS was applied at 0.31 and 0.63 mM in presence of laccase, L_{I} content significantly decreased from 168.6 to 157.7 and 157.2 to 138.4 mg·g⁻¹ when measured after 24 and 72 h (Table 3). Similarly, L_{T} content in sweet sorghum decreased from 210.6 to 201.4, 209.9 to 197.7, and 204.2 to 182.4 mg·g⁻¹ when measured after 24 and 72 h at ABTS concentration of 0.25, 0.31, and 0.63 mM, respectively (Table 3). In switchgrass biomass a significant duration effect was observed for L_{I} and L_{T} content at ABTS concentration of 0.63 and 1.25 mM (Table 4). A reduction of 14.7 and 16.1 mg·g⁻¹ and 17.4 and 18.7 mg·g⁻¹ was obtained for L_{I} and L_{T} content in switchgrass when measured after 24 and 72 h at ABTS concentration of 0.63 and 1.25 mM (Table 4).

### Laccase-HBT Mediator System

Laccase-HBT system had significant effects (P≦0.001) on L_{S}, L_{I} and L_{T} content of sweet sorghum and switchgrass. After 24 h of treatment with laccase along with HBT mediator up to concentration of 0.63 mM, a reduction in the range of 1.5-2.2 mg·g⁻¹ in L_{S} content was obtained in sweet sorghum (FIG. 2.3A). No reduction in L_{S} content was observed with higher HBT content (FIG. 2.3A). HBT application without laccase had no significant effect on the L_{S} content when compared to control (FIG. 2.3A). Extractive-free acid-insoluble content of sweet sorghum was significantly lowered by 13.3 (8%), 27.4 (16%), 31.8 (18%), 41.7 (24%), 49.2 (28%), and 55.1 (31%) mg·g⁻¹ with HBT concentration of 0.13, 0.25, 0.31, 0.63, 1.25, and 1.88 mM, respectively when applied along with laccase (FIG. 2.3B). A slight reduction in L_{I} content was observed when sweet sorghum was treated with 0.63 mM concentration of HBT without laccase. A 25.5% reduction in total lignin content of sweet sorghum was observed when HBT was applied at concentration of 1.88 mM along with laccase enzyme (Table 2).

A significant reduction in L_{S} content in switchgrass was observed in the range of 6.5-6.8 mg·g⁻¹ when treated with HBT alone at all the concentrations (FIG. 2.4A). When HBT was applied with laccase L_{S} content was lowered by 7.2, 7.4, and 8.1 mg·g⁻¹ at HBT concentration of 0.13, 0.25, and 0.31, respectively. However, at HBT concentration of 1.25 and 1.88 mM along with laccase, a significant increase in L_{S} content was observed (FIG. 2.4A). No effect of HBT alone was observed on L_{I} content of switchgrass. However, L_{I} content was significantly lowered by 9.7, 11.9, 15.9, 18.5, and 25.1 mg·g⁻¹ at HBT concentration of 0.25, 0.31, 0.63, 1.25, and 1.88, respectively when compared to control (FIG. 2.4B). Similarly, L_{T} content was lowered by 9.9 (4%), 16.8 (6%), 20.1 (8%), 19.5 (8%), 15.9 (6%), and 23.9 (9%) mg·g⁻¹ when compared to control at HBT concentration of 0.13, 0.25, 0.31, 0.63, 1.25, and 1.88 mM, respectively when applied with laccase enzyme (Table 2).

A slight but significant decrease in L_{S} content was observed for sweet sorghum between 24 and 72 h at HBT concentration of 0.31 mM (Table 3). Similarly, L_{I} content of sweet sorghum decreased by 14.5 and 7.5 mg·g⁻¹ and L_{T} content decreased by 14.1 and 7.8 mg·g⁻¹ when measured between 24 and 72 h at HBT concentration of 0.31 and 0.63 mM (Table 3). However, for switchgrass not significant duration effect was observed for L_{S}, L_{I}, and L_{T} except a slight reduction in L_{S} content with duration at HBT concentration of 0.63 mM (Table 4).

### Laccase-VA Mediator System

A significant treatment effect (P≦0.001) of laccase-VA system was observed on L_{S}, L_{I} and L_{T} content of sweet sorghum and switchgrass. After 24 h of laccase-VA treatment of sweet sorghum, no effect on L_{S} content was observed with application of VA without laccase. However, a 1.9-7.5 mg·g⁻¹ (5-20%) increase in L_{S} content was observed over control when VA was applied along with laccase at concentrations 0.63 to 1.88 mM (FIG. 2.5A). Extractive-free acid-insoluble lignin (L_{I}) content of sweet sorghum biomass was not affected when reaction mixture consisted of mediator without laccase enzyme. In the presence of laccase, L_{I} content of sweet sorghum was lowered by 38.9 (22%), 45.5 (26%), 53.6 (30%), and 52.6 (29%) mg·g⁻¹ when compared to control at VA concentration of 0.31, 0.63, 1.25, and 1.88 mM, respectively (FIG. 2.5B). At the same VA concentrations with laccase in the reaction mixture, reduction in L_{T} content were 38.5, 46.7, 50.7, and 45.1 mg·g⁻¹ representing 18, 22, 24, and 21% reduction, respectively (Table 2).

After 24 h of treatment of switchgrass biomass with VA without laccase, a significant decrease in L_{S} content in the range of 7.5-8.8 mg·g⁻¹ was observed when compared to the control (FIG. 2.6A). When applied along with laccase, a slight decrease in L_{S} content (21%) was observed at VA concentration of 0.31 mM when compared to control (FIG. 2.6A). At VA concentration of 0.63-1.88 mM, L_{S} content increased by 1.3-3.5 mg·g⁻¹ over the control (FIG. 2.6A). No effect of VA was observed on switchgrass biomass L_{I} content when treated without laccase enzyme (FIG. 2.6B). When applied along with laccase, a significant reduction in L_{I} content was observed for all the concentrations of VA (FIG. 2.6B). However, the maximum reduction in L_{I} content was 75 (34%) mg·g⁻¹ when compared to control at VA concentration of 0.63 mM (FIG. 2.6B). With increasing concentration of VA, the extent of L_{I} reduction was lowered. Similarly, the maximum reduction in extractive-free total lignin content in switchgrass amounted to 73.2 (28%) mg·g⁻¹ obtained when VA at 0.63 mM was applied along with laccase enzyme (Table 2). At higher VA concentrations, the impact on lignin reduction decreased significantly (Table 2).

A significant reduction in sweet sorghum L_{S} content was observed when reaction time for laccase-VA system was increased from 24 to 72 h at VA concentration of 0.31 and 0.63 mM (Table 3). However, no further reduction in L_{I} content of sweet sorghum was obtained with increase in reaction time (Table 3). An overall decrease of 20 mg·g⁻¹ in sweet sorghum L_{T} content was obtained with the duration of reaction at VA concentration of 0.31 mM (Table 3). No significant effect of laccase-VA system duration effect was observed for switchgrass L_{I} and L_{T} content at any VA concentration (Table 4). However, a reduction in L_{S} content of switchgrass was obtained with increased duration for VA concentration of 0.63, 1.25, and 1.88 mM (Table 4).

### Structural Sugars

Structural sugar content (S_{T}) in treated sweet sorghum biomass was lowered by 20-40 mg·g⁻¹ in comparison to control when ABTS was applied along with laccase enzyme at 0.63, 1.25, 1.88 mM concentration (Table 8.5). At the same ABTS concentrations a slight but non-significant reduction in structural sugar content was obtained (Table 8.5). However no further reduction in sugar content was observed for both species when reaction time increased from 24 h to 72 h (data not shown). A 24-34 and 23-31 mg·g⁻¹ reduction in structural sugar content in comparison to control was observed for sweet sorghum and switchgrass, respectively when treated with the laccase-HBT system consisting of HBT at 0.63, 1.25, and 1.88 mM. No reduction in sugar content was observed with increase in treatment duration from 24 h to 72 h at the same HBT concentrations. Laccase-VA system consisting of the same VA concentrations significantly reduced sugar content in sweet sorghum when compared to control at VA concentration of 1.88 mM (Table 5). However, S_{T} content in treated switchgrass was reduced at VA concentrations of 0.63 (62.6 mg·g⁻¹) and 1.25 mM (18.6 mg·g⁻¹) compared to the control.

### Dry Mass Loss

Weight loss in sweet sorghum and switchgrass was measured after the enzymatic treatments. A weight loss of 35-49, 70-78, and 63-69 mg·g⁻¹ in comparison to control were observed in sweet sorghum for ABTS, HBT, and VA mediator system, respectively (FIG. 2.7A). Similarly, 51-81, 53-56, and 56-63 mg·g⁻¹ loss in switchgrass weight was obtained when compared to control after treatment with ABTS, HBT, and VA mediator system, respectively (FIG. 2.7B). Loss in sweet sorghum and switchgrass weight after enzymatic treatment were calculated by addition of total lignin removal and structural sugar loss. Calculated and measured weight loss for both species is similar for HBT and VA mediator systems. However, for ABTS mediator system, calculated and measured weight loss in switchgrass are better related when weight loss due to acid-insoluble lignin is considered instead of the total lignin (FIG. 2.7B).

### DISCUSSION

Treatment of biomass with laccase without a mediator was not effective in removal of lignin from both biomass sources. Laccase-mediator system was effective in decreasing lignin content from the two biomass sources; however the efficacy of different mediators was different in different biomass species for L_{S} and L_{I}. The efficiency of laccase mediator system to remove lignin can be attributed to the high oxidation potential of the oxidized mediator and the small size of the mediators in comparison to laccase which makes it easier for them to reach deep within biomass structure to oxidize lignin bonds (Bourbonnais et al., 1997).

No significant reduction in L_{S} content was observed in sweet sorghum when ABTS concentration was 0.31 mM or less (FIG. 2.1A). However, in switchgrass a significant reduction was observed at the same concentrations (FIG. 2.2A). Optimal concentration for L_{I} removal was 1.25 and 1.88 mM for sweet sorghum and switchgrass, respectively. In sweet sorghum 8% total lignin reduction was obtained at 1.25 mM as compared to 14% in switchgrass at 1.88 mM concentration of ABTS when applied with laccase enzyme (FIG. 2.1B, FIG. 2.2B).

Optimum HBT concentration in the presence of laccase for L_{S} content reduction was up to 0.63 and 0.31 mM in sweet sorghum and switchgrass, respectively (FIG. 2.3A, FIG. 2.4A). Optimum reduction of L_{I} in both grass species was observed at 1.88 mM concentration in the presence of laccase. Laccase-HBT system was more effective in reducing L_{I} content in sweet sorghum (31%) as compared to switchgrass (12%) (FIG. 2.3B, FIG. 2.4B). Extractive-free total lignin content was lowered in sweet sorghum at 1.88 mM HBT along with laccase whereas L_{T} content in switchgrass decreased when HBT concentration in the reaction mixture was in the range of 0.25-1.88 mM along with laccase (Table 2).

When VA was applied along with laccase, an increase in L_{S} content was observed in sweet sorghum at concentration 0.63 mM and above (FIG. 2.5A). On the other hand, a reduction in switchgrass L_{S} content was observed at concentration of 0.31 mM and an increase in L_{S} content was obtained at 0.63 mM concentration or above (FIG. 2.6A). The optimum concentration of VA in laccase-VA system to lower the L_{I} and L_{T} content was 1.25 and 0.63 mM for sweet sorghum and switchgrass, respectively (Table 2, FIGS. 2.5B, 2.6B).

The different optimum concentrations of the three mediators on the same lignocellulosic biomass may stem from the difference in the mode of actions of these mediators. ABTS oxidizes lignin bonds by extraction of electrons whereas HBT and VA abstracts hydrogen atom for oxidation (Cantarella et al., 2003). Our results suggest that structure of the lignocellulosic biomass may also influence the extent of lignin removal (Chunxia et al., 2010).

Effect of enzymatic treatment duration on sweet sorghum and switchgrass varied with the choice of mediator. The duration effect of laccase-ABTS system was observed at different ABTS concentrations in the two biomass species. The effect was observed at 0.31 and 0.63 mM and 0.63 and 1.25 mM ABTS in sweet sorghum and switchgrass, respectively (Table 3, 4). It is seen that with higher ABTS concentration optimum lignin removal reached after 24 h and had no further duration effect, whereas with lower ABTS concentrations duration effect was shown, but the amount of delignification is not increased with increase in the reaction time. This may suggest that optimum amount of lignin removal in laccase-ABTS system can be achieved after 24 h with optimum mediator concentration.

The duration effect of laccase-HBT system on sweet sorghum was observed with HBT concentrations of 0.31 and 0.63 mM for L_{I} and L_{T} contents and with 0.31 mM HBT for L_{S} content (Table 3). However, no significant duration effect for L_{I} and L_{T} contents was observed at any HBT concentration in switchgrass biomass except a slight reduction in switchgrass L_{S} content was obtained at 72 h when compared to 24 h at 0.63 mM concentration (Table 4).

Laccase-VA system had no duration effect on total lignin content of switchgrass at different VA concentrations, while a slight reduction of L_{T} content in sweet sorghum was seen at VA concentration of 0.31 mM VA (Table 3, 4). This suggests that violuric acid as laccase mediator functions differently in different biomass species. The duration effect of VA on L_{S} content was observed at 0.31 and 0.63 mM for sweet sorghum and 0.63, 1.25, and 1.88 mM for switchgrass.

Different laccase mediators impacted the S_{T} content to different extents on sweet sorghum and switchgrass. Laccase-ABTS system reduced a S_{T} to a significant extent from the sweet sorghum but no significant reduction from switchgrass biomass. Laccase-HBT system impacted S_{T} on both biomass sources to the extent of 23-31 mg·g⁻¹. Laccase-VA system lowered the sugar content from sweet sorghum at 1.88 mM, but in switchgrass no S_{T} content was impacted at this concentration (Table 5).

### CONCLUSIONS

The accessibility of structural sugars in lignocellulosic biomass needs to be improved for enhancing bioethanol production efficiency. Structural sugar availability for fermentation can be increased by removing lignin protective matrix. The results from this experiment suggest the efficacy of laccase-mediator system in delignification of sweet sorghum and switchgrass. The ability of these mediators to remove lignin varied with biomass species. Lignin removal from sweet sorghum was in the range of 24-25.5% at 1.25 mM VA and 1.88 mM HBT in combination with laccase after 24 h treatment. In switchgrass, with application of laccase-ABTS system a 5-14% removal of lignin was observed and 28% lignin removal at 0.63 mM VA in combination with laccase after 24 h treatments.

References, each of which is incorporated herein by reference:

Abatzoglov, N., J. Bouchard, E. Chornet, and R. P. Overend. 1986. Dilute acid depolymerization of cellulose in aqueous phase: Experimental evidence of the significant presence of soluble oligomeric intermediates. Can. J. Chem. Eng. 64: 781-786.

Ayhan, D. 2007. Progress and recent trends in biofuels. Prog. Energ. Combust. 33: 1-18.

Blanchette, R. A. 1984. Screening wood decayed by white rot fungi for preferential lignin degradation. Appl. Environ. Microbiol. 48: 647-53.

Bouchard, J., N. Abatzoglou, E. Chornet, and R. P. Overend. 1989. Characterization of depolymerized cellulosic residues. Wood Sci. Technol. 23: 343-355.

Bouchard, J., R. P. Overend, E. Chornet, and M.-R Van Calsteren, 1992. Mechanism of dilute acid hydrolysis of cellulose accounting for its degradation in the solid state. J. Wood Chem. Technol. 12: 335-354.

Bourbonnais, R., and M. G. Paice. 1992. Demethylation and delignification of kraft pulp by Trametes versicolor laccase in the presence of 2,2′-azinobis-(3-ethylbenzthiazoline-6-sulphonate). Appl. Microb. Biotechnol., 36: 823-827.

Bourbonnais, R., M. G. Paice, B. Freiermuth, E. Bodie, and S. Borneman. 1997. Reactivities of various mediators and laccases with kraft pulp and lignin model compounds. Appl. Environ. Microbiol. 63(12): 4627-32.

Bourbonnais, R., M. G. Paice, I. D. Reid, P. Lanthier, and M. Yaghuchi. 1997. Lignin oxidation by laccase isozymes from Trametes versicolor and role of mediator 2,2′-Azinobis (3-Ethylbenzthiazoline-6-sulfonate) in kraft lignin depolymerization. Appl. Environ. Microbiol. 61: 1876-1880.

Cantarella, G., C. Galli, and P. Gentili. 2003. Free radical versus electron-transfer routes of oxidation of hydrocarbons by laccase/mediator systems: Catalytic or stoichiometric procedures. J. Mol. Catal. B-Enzym. 22: 135-144.

Cheng, K. K., J. A. Zhang, W. X. Ping, J. P. Ge, Y. J. Zhou, H. Z. Ling, and J. M. Xu. 2008. Sugarcane bagasse mild alkaline/oxidative pretreatment for ethanol production by alkaline recycle process. Appl. Biochem. Biotechnol. 15: 43-50.

Chunxia, Lu., H. Wang, L. Yuanming, and L. Guo. 2010. An efficient system for pre-delignification of gramineous biofuel feedstock in vitro: Application of a laccase from Pycnoporussanguineus H275. 45: 1141-1147.

Conner, A. H., B. F. Wood, C. G. Hill, and J. F. Harris. 1985. Kinetic model for the dilute sulfuric acid saccharification of lignocellulose. J. Wood Chem. Technol. 5: 461-489.

Conte, P., A. Maccotta, C. De Pasquale, S. Bubici, and G. Alonzo. 2009. Dissolution mechanism of crystalline cellulose in H₃PO₄ as assessed by high-field NMR spectroscopy and fast field cycling NMR relaxometry. J. Agric. Food Chem. 57: 8748-8752.

Crestini, C., and D. S. Argyropoulos. 1998. The early oxidative biodegradation steps of residual kraft lignin models with laccase. Bioorg. Med. Chem. 6: 2161-2169.

Fabbrini, M., C. Galli, and P. Gentili. 2002. Comparing the catalytic efficiency of some mediators of laccase. J. Mol. Catal. B-Enzym. 16: 231-240.

Fabbrini, M., C. Galli, P. Gentili, and D. Macchitella. 2001. An oxidation of alcohols by oxygen with the enzyme laccase and mediation by TEMPO. Tetrahedron Lett. 42: 7551-7553.

Fang, X., Y. Shen, J. Zhao, X. Bao, and Y. Qu. 2010. Status and prospect of lignocellulosic bioethanol production in China. Bioresour. Technol. 101: 4814-4819.

Fritz-Langhals, E., and B. Kunath. 1998. Synthesis of aromatic aldehydes by laccase-mediator assisted oxidation. Tetrahedron Lett. 39: 5955-5956.

Johannes, C., and A. Majcherczyk. 2000. Natural mediators in the oxidation of polycyclic aromatic hydrocarbons by laccase mediator systems. Appl. Environ. Microbiol. 66: 524-528.

Kang, K. H., J. Dec, H. Park, and J. M. Bollag. 2002. Transformation of the fungicide cyprodinil by a laccase of Trametes villosa in the presence of phenolic mediators and humic acid. Water Res. 36: 4907-4915.

Kaparaju, P., M. Serrano, A. B. Thomsen, P. Kongjan, and I. Angelidaki. 2009. Bioethanol, biohydrogen and biogas production from wheat straw in a biorefinery concept. Bioresour. Technol. 100: 2562-2568.

Kerr, A. J., and D. A. I. Goring. 1975. The ultrstructural arrangement of wood cell wall. Cellulose Chem. Technol. 9: 563-573.

Kersten, P. J., B. Kalyanaraman, K. E. Hammel, B. Reinhammar, and T. K. Kirk. 1990. Comparison of lignin peroxidase, horseradish peroxidase and laccase in the oxidation of methoxybenzenes. Biochem J. 268: 475-80.

Kim, Y.-J., and J. A. Nicell. 2006. Impact of reaction conditions on the laccase-catalyzed conversion of bisphenol A. Bioresour. Technol. 97: 1431-1442.

Kirk, T. K., W. J. Connors, R. D. Bleam, W. F. Hackett, an J. G. Zeikus. 1975. Preparation and microbial decomposition of synthetic [14C]ligins. Proc. Natl. Acad. Sci. U S A, 72(7): 2515-2519.

Kirk, T. K., W. J. Connors, and J. G. Zeikus. 1976. Requirement for a growth substrate during lignin decomposition by two wood-rotting fungi. Appl. Environ. Microbiol. 32: 192-194.

Li, J., G. Henriksson, and G. Gellerstedt. 2007. Lignin depolymerization/repolymerization and its critical role for delignification of aspen wood by steam explosion. Bioresour. Technol. 98: 3061-3068.

Matsushita, Y., T. Inomata, T. Hasegawa, and K. Fukushima. 2009. Solubilization and functionalization of sulfuric acid lignin generated during bioethanol production from woody biomass. Bioresour. Technol. 100: 1024-1026.

Mok, W. S., M. J. Antal, and G. Varhegyi. 1992. Productive and parasitic pathways in dilute acid-catalyzed hydrolysis of cellulose. Ind. Eng. Chem. Res. 31: 94-100.

NREL—National Renewable Energy Laboratory. 2008a. Preparation of Samples for Compositional Analysis. Available at: http://www.nrel.gov/biomass/pdfs/42620.pdf (Verified 01 July, 2012). NREL, Golden, Colo., USA.

NREL—National Renewable Energy Laboratory. 2008b. Determination of structural carbohydrates and lignin in biomass. Available at: http://www.nrel.gov/biomass/pdfs/42618.pdf (Verified 01 July, 2012). NREL, Golden, Colo., USA.

Potthast, A., T. Rosenau, C. L. Chen, and J. S. Gratzl. 1995. Selective enzymic oxidation of aromatic methyl groups to aldehydes. J. Org. Chem. 60: 4320-4321.

Qian, X., M. R. Nimlos, M. Davis, D. K. Johnson, and M. E. Himmel. 2005. Ab initio molecular dynamics simulations of β-d-glucose and β-d-xylose degradation mechanisms in acidic aqueous solution. Carbohydr. Res. 340: 2319-2327.

Ten Have, R., and P. J. M. Teunissen. 2001. Oxidative Mechanisms involved in lignin degradation by white-rot fungi. Chem. Rev. 101: 3397-3414.

Tsutsumi, Y., T. Haneda, and T. Nishida. 2001. Removal of estrogenic activities of bisphenol A and nonylphenol by oxidative enzymes from lignin-degrading basidiomycetes. Chemosphere. 42: 271-276.

Xiang, Q., Y. Y. Lee, and R.W. Torget. 2004. Kinetics of glucose decomposition during dilute-acid hydrolysis of lignocellulosic biomass. Appl. Biochem. Biotechnol. 113: 1127-1138.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to measurement techniques and the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

We claim the following:

1. A method of treating a biomass, comprising:
contacting a lignocellulosic biomass with an enzyme;
mixing the lignocellulosic biomass with the enzyme at about room temperature for a time period; and
modifying the content of lignin in a lignocellulosic biomass.

2. The method of claim 1, wherein the enzyme is selected from the group consisting of: laccase, lignin peroxidase, horseradish peroxidase, manganese peroxidase, tyrosinase, and a combination thereof.

3. The method of claim 2, wherein the enzyme is about 1/100 to 1/100,000,000 of the mass of the lignocellulosic biomass.

4. The method of claim 1, further comprising:
contacting the lignocellulosic biomass with a mediator; and
mixing the lignocellulosic biomass with the enzyme and the mediator at about room temperature for a time period.

5. The method of claim 4, wherein the mediator is selected from the group consisting of: catechol, guaiacol, ABTS, violuric acid, 1-hydroxy-benzotriazole (HBT), veratryl alcohol, and a combination thereof.

6. The method of claim 5, wherein the mediator is about 1/100 to 1/100,000,000 of the mass of the lignocellulosic biomass.

7. The method of claim 4, wherein the enzyme is selected from the group consisting of: laccase, lignin peroxidase, horseradish peroxidase, manganese peroxidase, tyrosinase, and a combination thereof wherein the mediator is selected from the group consisting of: catechol, guaiacol, ABTS, violuric acid, 1-hydroxy-benzotriazole (HBT), veratryl alcohol , and a combination thereof; and wherein the ratio enzyme, to the mediator, to the lignocellulosic biomass is about 1:1:100 to 1:100,000:100,000,000.

8. The method of claim 4, wherein modifying includes reducing the content of lignin in the lignocellulosic biomass relative the amount originally present in the lignocellulosic biomass.

9. The method of claim 8, wherein reducing includes reducing the lignin content by about 15% or more relative to the original lignin content.

10. The method of claim 9, wherein reducing includes substantially maintaining the cellulose and hemicellulose content while reducing the lignin content by about 25% or more relative to the original lignin content.

11. The method of claim 4, wherein modifying includes an enzymatic hydrolysis of the lignocellulosic biomass is about 35% or more after about a 24 hour or more time period relative to no treatment with the enzyme and the mediator.

12. The method of claim 1, wherein the lignocellulosic biomass is selected from the group consisting of: switch grass, sweet sorghum, miscanthus, corn stover, and pine wood.

13. The method of claim 1, wherein the time period is about 24 hours or more.

14. The method of claim 1, wherein the enzyme is laccase.

15. The method of claim 13, wherein the laccase is an isolated laccase.

16. A composition, comprising an enzyme and a mediator, wherein the enzyme is selected from the group consisting of: laccase, lignin peroxidase, horseradish peroxidase, manganese peroxidase, tyrosinase, and a combination thereof; wherein the mediator is selected from the group consisting of: catechol, guaiacol, ABTS, violuric acid, 1-hydroxy-benzotriazole (HBT), veratryl alcohol and a combination thereof.
